(19) United States
(12) Patent Application Publication
(10) Pub. No.: US 2009/0280167 A1
(43) Pub. Date: Nov. 12, 2009

(54) ENHANCEMENT OF DRUG THERAPY BY MIRNA

(75) Inventors: Vuong Trieu, Calabasas, CA (US); Larn Hwang, Arcadia, CA (US); Neil Desai, Los Angeles, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Abraxis BioScience, LLC, Los Angeles, CA (US)

(21) Appl. No.: 12/436,937

(22) Filed: May 7, 2009

Related U.S. Application Data
(60) Provisional application No. 61/051,309, filed on May 7, 2008.

(51) Int. Cl. A61K 31/7088; A61K 9/127; A61K 39/395; C07H 21/04; C12Q 1/68

(52) U.S. Cl. 424/450; 514/44 R; 424/133.1; 536/24.3; 435/6; 536/23.1; 536/23.2

(57) ABSTRACT

This invention provides methods and compositions for screening of microRNA capable of modulating gene expression in the apoptotic pathway in the presence of HSP90 inhibitor. The use of miRNA for enhancing the activity of therapeutic agents not limited to HSP90 inhibitor is also disclosed. The diagnostic use of miRNA for predicting response to therapy not limited to therapeutic agents is also disclosed. A method for the identification and therapeutic application of small molecules which are modulators of these nucleic acids are also included in this application

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Accordingly, there is a need to continue to develop new therapies and, in particular, new therapies that work well, if not synergistically, in conjunction with other agents and treatment.

Heat shock proteins (HSPs) are a class of chaperone proteins that are up-regulated in response to elevated temperature and other environmental stresses, such as ultraviolet light, nutrient deprivation, and oxygen deprivation. HSPs act as chaperones to other cellular proteins (called “client” proteins) and facilitate their proper folding and repair of client proteins. There are several known families of HSPs, each having its own set of client proteins. The HSP90 family is one of the most abundant HSP families, accounting for about 1-2% of proteins in a cell that is not under stress and increasing to about 4-6% in a cell under stress. Inhibition of HSP90 results in degradation of its client proteins via the ubiquitin proteasome pathway. Unlike other chaperone proteins, the client proteins of HSP90 are mostly protein kinases or transcription factors involved in signal transduction, and a number of its client proteins have been shown to be involved in the progression of cancer. Thus, the inhibition of HSP90 is a promising avenue for the treatment of cancer and other diseases.

17-AAG is an ansamycin antibiotic which binds to HSP90 (Heat Shock Protein 90) and alters it function. Specifically, 17-AAG binds with a high affinity into the ATP binding pocket in HSP90 and induces the degradation of proteins that require this chaperone for conformational maturation.

Rapamycin is a drug used to prevent the rejection of organ and bone marrow transplants by the body. Rapamycin is an antibiotic that blocks a protein involved in cell division and inhibits the growth and function of certain T cells of the immune system involved in the body's rejection of foreign tissues and organs. It is a type of immunosuppressant and a type of serine/threonine kinase inhibitor. Rapamycin is also called sirolimus.

The platinum-based chemotherapy agents (“platinum agents”) include, e.g., oxaliplatin, cisplatin and carboplatin. The platinum agents class of drugs crosslink DNA in several different ways, interfering with cell division by mitosis. The damaged DNA elicits DNA repair mechanisms, which in turn activate apoptosis when repair proves impossible. In addition, it has been postulated that platinum agents also react with cellular proteins, particularly HMG domain proteins, further interfering with mitosis.

Paclitaxel is a mitotic inhibitor used in cancer chemotherapy. Paclitaxel is also used for the prevention of restenosis. Together with docetaxel, it forms the drug category referred to as the taxanes. Taxanes work by interfering with normal microtubule breakdown during cell division.

Notably, the HSP inhibitors, platinum agents, and taxanes are members of different drug classes and have widely different mechanism of action.

### BRIEF SUMMARY OF THE INVENTION

The invention provides methods for enhancing the activity of a therapeutic agent in an organism afflicted with cancer, neurodegenerative diseases, restenosis or proliferative cellular diseases comprising administering a therapeutically effective amount of an miRNA before, during or after administering another therapeutic agent. In a preferred embodiment the methods and compositions of the invention are directed to enhancing the activity of the HSP90 inhibitor 17-AAG.

Methods and composition in a accord with the invention include, e.g., wherein the miRNA comprises one or more the following RNA sequences:

| | |
|---|---|
| miR145 | |
| (GUCCAGUUUUCCCAGGAAUCCCUU), | (SEQ ID NO: 1) |
| miR4543p | |
| (UAGUGCAAUAUUGCUUAUAGGGUUU), | (SEQ ID NO: 2) |
| miR519a | |
| (AAAGUGCAUCCUUUUAGAGUGUUAC), | (SEQ ID NO: 3) |
| miR520c | |
| (AAAGUGCUUCCUUUUAGAGGGUU), | (SEQ ID NO: 4) |
| miR520d | |
| (AAAGUGCUUCUCUUUGGUGGGUU), | (SEQ ID NO: 5) |
| miR-425-3p | |
| (AUCGGGAAUGUCGUGUCCGCC), | (SEQ ID NO: 6) |
| miR-495 | |
| (AAACAAACAUGGUGCACUUCUUU), | (SEQ ID NO: 7) |
| miR-572 | |
| (GUCCGCUCGGCGGUGGCCCA), | (SEQ ID NO: 8) |
| and | |
| miR-661 | |
| (UGCCUGGGUCUCUGGCCUGCGCGU). | (SEQ ID NO: 9) |

Alternatively, in other embodiments the miRNA comprises one or more of SEQ ID NOs:10-35.

In particular, the invention provides methods and compositions wherein the miRNA is selected from the group consisting of: miR454-3p (UAGUGCAAUAUUGCUUAUAGGGUUU) (SEQ ID NO:2), miR520c (AAAGUGCUUCCUUUUAGAGGGUU) (SEQ ID NO:4), complements thereof and combinations thereof, and the therapeutic agent is 17-AAG, oxaliplatin or a combination thereof.

Additionally, the invention provides methods and compositions wherein the miRNA is selected from the group consisting of:

miR-425-3p (AUCGGGAAUGUCGUGUCCGCC) (SEQ ID NO:6),
miR-495 (AAACAAACAUGGUGCACUUCUUU) (SEQ ID NO:7),
miR-572 (GUCCGCUCGGCGGUGGCCCA) (SEQ ID NO:8),
miR-661 (UGCCUGGGUCUCUGGCCUGCGCGU) (SEQ ID NO:9), complements thereof and combinations thereof, and the therapeutic agent is paclitaxel.

Accordingly, the invention provides methods for predicting response to therapy with a HSP90 inhibitor, microtubule inhibitor or mitotic inhibitor comprising: (a) providing a biological sample of diseased tissue; (b) measuring the level of a RNA in biological sample of diseased tissue wherein the RNA measured is comprised of the following:

| | |
|---|---|
| miR145 | |
| (GUCCAGUUUUCCCAGGAAUCCCUU), | (SEQ ID NO: 1) |
| miR454-3p | |
| (UAGUGCAAUAUUGCUUAUAGGGUUU), | (SEQ ID NO: 2) |
| miR519a | |
| (AAAGUGCAUCCUUUUAGAGUGUUAC), | (SEQ ID NO: 3) |
| miR520c | |
| (AAAGUGCUUCCUUUUAGAGGGUU), | (SEQ ID NO: 4) |
| miR520d | |
| (AAAGUGCUUCUCUUUGGUGGGUU), | (SEQ ID NO: 5) |
| miR-425-3p | |
| (AUCGGGAAUGUCGUGUCCGCC), | (SEQ ID NO: 6) |
| miR-495 | |
| (AAACAAACAUGGUGCACUUCUUU), | (SEQ ID NO: 7) |
| miR-572 | |
| (GUCCGCUCGGCGGUGGCCCA), | (SEQ ID NO: 8) |
| miR-661 | |
| (UGCCUGGGUCUCUGGCCUGCGCGU),; | (SEQ ID NO: 9) |

(ii) an RNA complementary of (i); and

(iii) an RNA with a sequence at least about 81% identical to 21 contiguous nucleotides of (i) or (ii); (c) comparing the level of RNA from (b) in diseased tissue with the level of the same RNA in a control wherein a level of the nucleic acid higher than a control is indicative response to the therapy and lower than that of control is indicative of non-response to the therapy. Alternatively, in other embodiments the miRNA comprises one or more of SEQ ID NOs:10-35.

The invention provides isolated nucleic acids comprising a vector with one or more in vivo expression control elements operatively linked to a reporter gene, wherein said reporter gene is upstream of all or a portion of a 3′ untranslated region of a target gene (whose expression level is to be modulate, either increasing or decreasing its level), wherein upon transfection of the isolated nucleic acid into eukaryotic cells, the transfected cells express an mRNA encoding the reporter upstream of the 3′ untranslated region. Notably, exogenous miRNA provided in accordance with the invention may also interact with inhibitory endogenous miRNA to increase expression of the target gene.

Isolated nucleic acids in accordance with the invention include, e.g., wherein a vector selected from the group consisting of a plasmid, cosmid, phagemid, virus, and artificial chromosome. Isolated nucleic acids in accordance with the invention include, e.g., wherein the one or more in vivo expression control elements are selected from the group consisting of a promoter, enhancer, RNA splicing signal, and combinations thereof.

In preferred embodiments the reporter gene encodes a luciferase protein and the target gene is CD44, CDC27, MAPK activated kinase 2, PAR4 or PKC gamma and the 3′ untranslated region is from CD44, CDC27, MAPK activated kinase 2, PAR4 or PKC gamma.

The invention provides further provides methods of identifying expression modulators of a target gene comprising:
(a) transfecting eukaryotic cells with an isolated nucleic acid comprising one or more in vivo expression control elements operatively linked to a reporter gene which is cloned upstream of all or a portion of a 3′ untranslated region of a target gene, wherein the in vivo expression control elements result the production of an mRNA encoding the reporter upstream of the target gene 3′ untranslated region, and
(b) transfecting other eukaryotic cells with isolated nucleic acid comprising said one or more in vivo expression control elements operatively linked to said reporter gene, wherein the expression control elements result in the transcription of an mRNA encoding the reporter molecule,
(c) contacting and mock-contacting the transfected cells from (a) and (b) with a candidate expression modulator, and
(d) comparing the reporter gene activity in the transfected cells from (a) and (b) with and without contacting the transfected cells with candidate expression modulator.

Embodiments of the methods of claimed, include wherein the method further comprises the co-transfection of the cells in (a) and (b), with a second report construct expressing a second reporter for the normalization the data compared in (d). In preferred embodiments of the invention the target gene CD44, CDC27, MAPK activated kinase 2, PAR4 or PKC gamma. The method also provides for mutating the CD44, CDC27, MAPK activated kinase 2, PAR4, PKC gamma 3′ untranslated region in the reporter expression construct, transfecting said mutated reporter expression construct into eukaryotic cells, and comparing the reporter gene activity resulting from expression of the mutated and unmutated reporter expression constructs with and without contacting the transfected cells with candidate expression modulator.

Accordingly, the invention provides kits for the identification of target gene expression modulators comprising:
(a) first isolated nucleic acid with a first set of one or more in vivo expression control elements operatively linked to a first reporter gene which is cloned upstream of all or a portion of a 3′ untranslated region of a target gene, wherein upon transfection of said first isolated nucleic acid into eukaryotic cells, the first set of in vivo expression control elements result the production of an mRNA encoding the first reporter upstream of the a 3′ untranslated region of a target gene, such as, e.g., CD44, CDC27, MAPK activated kinase 2, PAR4 or PKC gamma;
(b) a second isolated nucleic acid comprising said the set of in vivo expression control elements from (a) operatively linked to said first reporter gene, wherein upon transfection of said second isolated nucleic acid into eukaryotic cells, the in vivo expression control elements result in the transcription of an mRNA encoding said first reporter molecule; and
(c) a third isolated nucleic acid comprising a second set of one or more in vivo expression control elements operatively linked to a second reporter gene, wherein upon transfection of the isolated nucleic acid into eukaryotic cells, said second set of in vivo expression control elements result in the expression of said second reporter.

In yet another embodiment, the invention provides for isolated nucleic acids comprising a miRNA, wherein when the miRNA is administered to mammalian cells and the mammalian cells are then exposed or contacted with to a therapeutic agent, the mammalian cells produce 485/538 nm ratio of at least about 200, preferably at least about 225, more preferably at least about 250, most preferably at least about 275 in an Apo-ONE® Homogeneous Caspase-3/7 Assay (Promega, Madison, Wis.) or other suitable assay for the quantification of apoptosis. Accordingly, the invention also provides for isolated nucleic acids capable of expressing a transcripts comprising a miRNAs, wherein when the miRNAs expressed in mammalian cells and the mammalian cells are then exposed to or contacted with a therapeutic agent, the mammalian cells produce 485/538 nm ratio of at least about 200, preferably at least about 225, more preferably at least about 250, most preferably at least about 275 in an Apo-ONE® Homogeneous Caspase-3/7 Assay (Promega, Madison, Wis.) or other suitable assay for the quantification of apoptosis. Suitable therapeutic agents for use in accordance with this aspect of the invention include, e.g., 17-AAG, oxaliplatin, paclitaxel and combinations thereof.

Further, the invention provides methods for enhancing the activity of a therapeutic agent in an organism afflicted with cancer, neurodegenerative diseases, restenosis or proliferative cellular diseases comprising administering a therapeutically effective amount of an miRNA before, during or after administering another therapeutic agent. In a preferred embodiment the methods and compositions of the invention are directed to enhancing the activity of the rapamycin. In such an embodiment, the invention provides for methods of enhancing, potentiating or increasing the activity of rapamycin comprising the expression of miRNAs comprising one or more of the sequences:

### CCAGUAUUAACUGUGCUGCUGA (SEQ ID NO:36),

### AAGUGUGCAGGGCACUGGU (SEQ ID NO:37), and

AAGGAGCUUACAAUCUAGCUGGG (SEQ ID NO:38) and combinations thereof.

### BRIEF DESCRIPTION OF THE SEVERAL VIEWS OF THE DRAWING(S)

FIG. 1 depicts the dose-response curve for the apoptotic activity of HSP90 inhibitor 17-AAG.

FIG. 2 depicts the dose-response curve for the down regulation of Her2 following inhibition of HSP90 by 17-AAG.

FIG. 3 depicts Her2's internalizalization and degradation following treatment with the HSP90 inhibitor 17-AAG.

FIG. 4 depicts the inhibition 17-AAG induced apoptotic activity by Velcade.

FIG. 5 is a tabular presentation of the miRNAs that induce apoptosis synergistically with a 1:800 dilution of 17-AAG. Dark background, best candidates at 1:800 and 1:3200. Light background, candidates at 1:800 only.

FIG. 6 is a tabular presentation of the miRNAs that induce apoptosis synergistically with a 1:3200 dilution of 17-AAG. Dark background, best candidates at 1:800 and 1:3200. Light background, candidates at 1:800 only.

FIG. 7 depicts the 17-AAG concentration dependency of apoptosis induction by the identified miRNAs. 1E2, mir-145 (SEQ ID NO:1); 3F6, mir-454 (SEQ ID NO:2); 4C6, mir-519 (SEQ ID NO:3); 4D4-520c (SEQ ID NO:4); 4D5, mir-520d (SEQ ID NO:5); 17AAG, no miRNA baseline activity.

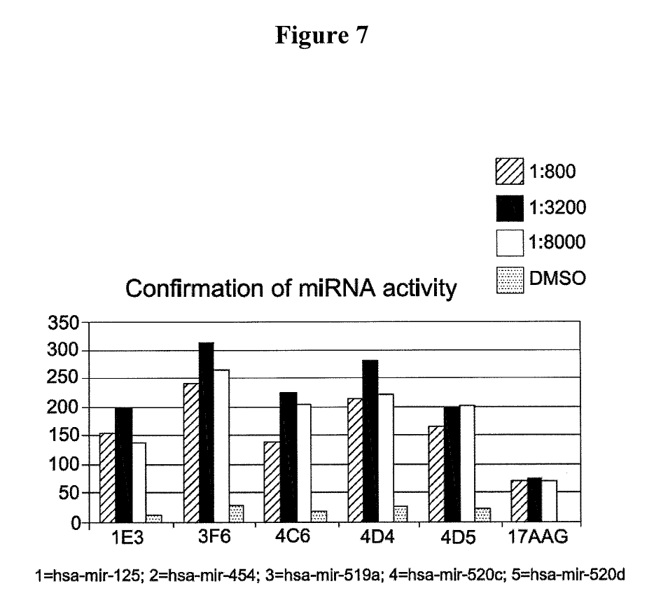

FIG. 8 presents the relationship and sequence alignment of the five miRNAs showing that has-mir-145 belong to a distinct class.

FIG. 9 shows the miRNA concentration dependency of the induction of apoptosis 17-AAG/miRNA. A, with a therapeutic agent; B, without a therapeutic agent.

FIG. 10 shows antibody microarray results.

FIG. 11 shows the enhancement of oxaliplatin activity by miRNAs.

FIG. 12 shows the enhancement of paclitaxel activity by miRNAs.

### DETAILED DESCRIPTION OF THE INVENTION

### I. Definitions

As used herein the term “administer” refers to the delivery to an organism of a therapeutic agent, e.g., a miRNA, such that the agent will contact and if necessary for function, enter diseased cells. In the case of a vector, expression of a miRNA in the diseased cells will also result from the administration. Many methods of administration are known to those of ordinary skill in the art.

As used herein the term “enhancing the activity of a therapeutic agent” means causing a significant change in the activity the therapeutic agent (e.g., a change of at least about 10%, at least about 20%, at least about 25%, at least about 33%, at least about 50%, at least about 100%, at least about 2 fold, at least about, 3 fold at least, about 10 fold, at least about 100 fold or more.) The enhancement may be manifest by the ability to reduce the dose, reduce the side effects or shorten the course of therapy.

As used herein the term “nucleic acid” refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art. Thus, the term nucleic acid also encompasses nucleic acids with substitutions or modifications, such as in the bases and/or sugars.

As used herein, the term “microRNA” (or miRNA) refers to any type of interfering RNA, including but not limited to, endogenous microRNA and artificial microRNA. Endogenous microRNA are small RNAs naturally present in the genome which are capable of modulating the productive utilization of mRNA. The term “artificial” or “synthetic” microRNA includes any type of RNA sequence, other than endogenous microRNA, which is capable of modulating the productive utilization of mRNA.

“MicroRNA flanking sequence” as used herein refers to nucleotide sequences including microRNA processing elements. MicroRNA processing elements are the minimal nucleic acid sequences which contribute to the production of mature microRNA from precursor microRNA. Precursor miRNA termed pri-miRNAs are processed in the nucleus into about 15-70 nucleotide pre-miRNAs, which fold into imperfect stem-loop structures.

The microRNA flanking sequences may be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. The artificial microRNA flanking sequences may be flanking sequences found naturally in the context of other microRNA sequences. Alternatively they may be composed of minimal microRNA processing elements which are found within naturally occurring flanking sequences and inserted into other random nucleic acid sequences that do not naturally occur as flanking sequences or only partially occur as natural flanking sequences.

The microRNA flanking sequences within the precursor microRNA molecule may flank one or both sides of the stem-loop structure encompassing the microRNA sequence. Preferred structures have flanking sequences on both ends of the stem-loop structure. The flanking sequences may be directly adjacent to one or both ends of the stem-loop structure or may be connected to the stem-loop structure through a linker, additional nucleotides or other molecules.

As used herein a “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures and terms are well known in the art. The actual primary sequence of nucleotides within the stem-loop structure is not critical as long as the secondary structure is present. As is known in the art, the secondary structure does not require exact base-pairing. Thus, the stem may include one or more base mismatches. Alternatively, the base-pairing may not include any mismatches.

A DNA isolate is understood to mean chemically synthesized DNA, cDNA or genomic DNA with or without the 3′ and/or 5′ flanking regions. DNA encoding miRNA can be obtained from other sources by a) obtaining a cDNA library from cells containing mRNA, b) conducting hybridization analysis with labeled DNA encoding miRNA or fragments thereof (usually, greater than 100 bp) in order to detect clones in the cDNA library containing homologous sequences, and c) analyzing the clones by restriction enzyme analysis and nucleic acid sequencing to identify full-length clones.

As used herein nucleic acids and/or nucleic acid sequences are homologous when they are derived, naturally or artificially, from a common ancestral nucleic acid or nucleic acid sequence. Homology is generally inferred from sequence identity between two or more nucleic acids or proteins (or sequences thereof). As used herein two nucleic acids and/or nucleic acid sequences, including miRNAs, are “identical” if they have the same nucleotide at each corresponding position in the two sequences, wherein for the purposes of this analysis uracil and thymidine are treated equivalently. Two sequences have a percent identity based on the number of identical nucleotides they share when the sequences are aligned by a suitable algorithm such as b12seq (Tatiana A. Tatusova, Thomas L. Madden (1999), “Blast 2 sequences—a new tool for comparing protein and nucleotide sequences”, FEMS Microbiol Lett. 174:247-250) which is publicly available through the National Center for Biotechnology Information. Two sequences are “complementary” if they can base pair at all nucleotides. The percent complementarity is based on the percent of nucleotides in each strand that can base pair with the other sequence when the sequences are aligned for base pairing.

As used herein a “therapeutic agent” refers to any suitable prior art drug or other treatment used to treat mammalian diseases or conditions. For example, azilect, L-dopa/carbidopa, mirapex or trihexyphenidyl for Parkinson's disease.

By “isolated nucleic acid” it is meant removed from its natural state and suitably pure for cloning.

### II. General Principles

A. Heat Shock Protein 90 (HSP90)

HSP90 has been shown by mutational analysis to be necessary for the survival of normal eukaryotic cells. In addition, HSP90 is over expressed in many tumor types indicating that it may play a significant role in the survival of cancer cells and that cancer cells may be more sensitive to inhibition of HSP90 than normal cells. For example, cancer cells typically have a large number of mutated and overexpressed oncoproteins that are dependent on HSP90 for folding. Examples of HSP90 client proteins that have been implicated in the progression of cancer include Her-2, c-Kit, c-Met, Akt kinase, Cdk4/cyclin D complexes, Raf-1 v-src, BCR-ABL fusion protein, steroid hormone receptors, p53 and Hif-1. In addition, because the environment of a tumor is typically hostile due to hypoxia, nutrient deprivation, acidosis, etc., tumor cells may be especially dependent on HSP90 for survival.

Thus, inhibition of HSP90 has promise for developing new cancer therapies. The inhibition of HSP90 causes simultaneous inhibition of a number of oncoproteins, as well as hormone receptors and transcription factors making it an attractive target for an anti-cancer agent.

HSP90 inhibitors may have utility in the treat of other diseases. For example, the HSP inhibitor, —17AAG, causes degradation of polyglutamine (polyQ)-expanded androgen receptor (AR) which is a pathogenic gene product in the neurodegenerative disease spinal and bulbar muscular atrophy (Waza M et al., 2006, Alleviating neurodegeneration by an anticancer agent. Annals of the New York Academy of Sciences 1086: 21-34).

B. Micro RNAs

Micro RNAs (referred to as “miRNAs”) are small non-coding RNAs, belonging to a class of regulatory molecules found in plants and animals that control gene expression by binding to complementary sites on target messenger RNA (mRNA) transcripts. miRNAs are generated from large RNA precursors (termed pri-miRNAs) that are processed in the nucleus into approximately 70 nucleotide pre-miRNAs, which fold into imperfect stem-loop structures (Lee, Y., et al., Nature (2003) 425(6956):415-9). The pre-miRNAs undergo an additional processing step within the cytoplasm where mature miRNAs of 18-25 nucleotides in length are excised from one side of the pre-miRNA hairpin by an RNase III enzyme, Dicer (Hutvagner, G., et al., Science (2001) 12:12 and Grishok, A., et al., Cell (2001) 106(1):23-34). mRNAs have been shown to regulate gene expression in two ways. First, miRNAs that bind to protein-coding mRNA sequences that are exactly complementary to the miRNA induce the RNA-mediated interference (RNAi) pathway. Messenger RNA targets are cleaved by ribonucleases in the RISC complex. This mechanism of miRNA-mediated gene silencing has been observed mainly in plants (Hamilton, A. J. and D. C. Baulcombe, Science (1999) 286(5441):950-2 and Reinhart, B. J., et al., MicroRNAs in plants. Genes and Dev. (2002) 16:1616-1626), but an example is known from animals (Yekta, S., I. H. Shih, and D. P. Bartel, Science (2004) 304(5670):594-6). In the second mechanism, miRNAs that bind to imperfect complementary sites on messenger RNA transcripts direct gene regulation at the posttranscriptional level but do not cleave their mRNA targets. mRNAs identified in both plants and animals use this mechanism to exert translational control of their gene targets (Bartel, D. P., Cell (2004) 116(2):281-97).

It is therefore an object of the present invention to provide naturally occurring miRNAs in combination therapy to enhance the therapeutic activity of any therapeutic agents in general. Herein a method for enhancing the activity of therapeutic agents with the HSP90 inhibitor is presented to demonstrate the method. Other therapeutic agents are suitable for activity enhancement include, e.g., oxaliplatin and paclitaxel.

It is further an object of the present invention to provide naturally occurring nucleic acids for treatment or prophylaxis of one or more symptoms of cancer or proliferative diseases which are dependent on or caused by HSP90 dysregulation.

It is further an object of the present invention to use the detection of miRNA as diagnostic for response to the therapy either as combination therapy with the miRNA or as single therapy with the therapeutic agent such as HSP90 inhibitor.

To meet these and other objective, the invention provides methods and composition for enhancing the activity of a therapeutic agent, such as the HSP inhibitor 17-AAG, in an organism afflicted with cancer, neurodegenerative diseases, restenosis or proliferative cellular diseases comprising administering an effective amount of an miRNA before, during or after administering the therapeutic agent.

Accordingly, the invention provides for an miRNA that can be either synthetic or encoded by an isolated nucleic acid. miRNA in accordance with the invention include, e.g., pri-miRNA, pre-miRNA, mature miRNA, ds miRNA and fragments or variants thereof. The miRNA can be chemically modified RNA, for example, it can be modified with a chemical mioety selected from the group consisting of phosphorothioate, boranophosphate, 2′-O-methyl, 2′-fluoro, terminal inverted-dT bases, PEG, and combinations thereof. The miRNA may be administered in accordance with the invention as a naked RNA, i.e., saline or D5, or in a cationic liposome, neutral liposome, polymer-based nanoparticle, cholesterol conjugate, cyclodextran complex, polyethylenimine polymer or a protein complex. The miRNA can be administered in accordance with the invention directly to the diseased tissue, intravenously, subcutaneously, intramuscularly, nasally, intraperitonealy, vaginally, anally orally, intraocularly or intratechally. See, e.g., Fougerolles, Human Gene Therapy (2008) 19:125-132; Behlke, Molecular Therapy (2006) 13(4): 644-670.

The invention also provides methods and therapeutic compositions comprising miRNAs for enhancing the activity of a therapeutic agent in an organism afflicted with cancer, neurodegenerative diseases, restenosis or proliferative cellular diseases comprising an effective amount of an miRNA or a vector that expresses an effective amount of an miRNA before (including, e.g. about 8 hours, about 12 hours, about 1 day, about 2 days, about 3 days, about 1 week, about 2 weeks, about 1 month or more before), during (including, e.g. simultaneously, with about 10 minutes, within about 30 minutes, within about 1 hour, within about 2 hours, within about 8 hours, within about 12 hours, within about 1 day) or after (including, e.g. about 8 hours, about 12 hours, about 1 day, about 2 days, about 3 days, about 1 week, about 2 weeks, about 1 month or longer after) administering the therapeutic agent.

In preferred embodiments, the invention provides isolated nucleic acids including those that comprise a sequence of any of the following miRNAs:

miR145 (GUCCAGUUUUCCCAGGAAUCCCUU) (SEQ ID NO:1),
miR454-3p (UAGUGCAAUAUUGCUUAUAGGGUUU) (SEQ ID NO:2),
miR519a (AAAGUGCAUCCUUUUAGAGUGUUAC) (SEQ ID NO:3),
miR520c (AAAGUGCUUCCUUUUAGAGGGUU) (SEQ ID NO:4),
miR520d (AAAGUGCUUCUCUUUGGUGGGUU) (SEQ ID NO:5),
miR-425-3p (AUCGGGAAUGUCGUGUCCGCC) (SEQ ID NO:6),
miR-495 (AAACAAACAUGGUGCACUUCUUU) (SEQ ID NO:7),
miR-572 (GUCCGCUCGGCGGUGGCCCA) (SEQ ID NO:8),
miR-661 (UGCCUGGGUCUCUGGCCUGCGCGU) (SEQ ID NO:9), the complement thereof, or a sequence at least 81% identical, preferably at least 95% identical to 18 contiguous nucleotides thereof. Alternatively, in other embodiments the miRNA comprises one or more of SEQ ID NOs:10-35

A probe comprising the nucleic acid or a peptide nucleic acid complementary to miRNAs is also provided. A composition comprising the probe is also provided. A biochip comprising the probe is also provided.

The invention further provides a biological sample may be assayed for the level of a nucleic acid may be measured. The nucleic acid may comprise a sequence of any of miRNAs: miR145 (GUCCAGUUUUCCCAGGAAUCCCUU) (SEQ ID NO:1), miR454-3p (UAGUGCAAUAUUGCUUAUAGGGUUU) (SEQ ID NO:2), miR519a (AAAGUGCAUCCUUUUAGAGUGUUAC) (SEQ ID NO:3), miR520c (AAAGUGCUUCCUUUUAGAGGGUU) (SEQ ID NO:4), miR520d (AAAGUGCUUCUCUUUGGUGGGUU) (SEQ ID NO:5). The nucleic acid may also comprise a sequence at least about 81% identical, preferably at least 95% identical to about 18 contiguous nucleotides of any of listed miRNAs. A level of the nucleic acid higher than that of a control may be indicative response to the therapy and lower than that of control indicative of non-response to the therapy—in this example being HSP90 inhibitors such as 17-AAG, oxaliplatin or a combination thereof.

The invention further provides a biological sample may be provided from which the level of a nucleic acid may be measured. The nucleic acid may comprise a sequence of any of miRNAs: miR-425-3p (AUCGGGAAUGUCGUGUCCGCC) (SEQ ID NO:6),

miR-495 (AAACAAACAUGGUGCACUUCUUU) (SEQ ID NO:7),
miR-572 (GUCCGCUCGGCGGUGGCCCA) (SEQ ID NO:8),
miR-661 (UGCCUGGGUCUCUGGCCUGCGCGU) (SEQ ID NO:9). The nucleic acid may also comprise a sequence at least about 81% identical, preferably at least 95% identical to about 18 contiguous nucleotides of any of listed miRNAs. A level of the nucleic acid higher than that of a control may be indicative response to the therapy and lower than that of control indicative of non-response to the therapy—e.g., for the sequences of miR-425-3p (SEQ ID NO:6), miR-495 (SEQ ID NO:7), miR-572(SEQ ID NO:8), and miR-661(SEQ ID NO:9) for paclitaxel.

The invention also provides a method for identifying a compound that modulates expression of a cancer-associated miRNA: (a) providing a cell that is capable of expressing a nucleic acid according to claim 1; (b) contacting the cell with a candidate modulator; and (c) measuring the level of expression of the nucleic acid, wherein a difference in the level of the nucleic acid compared to a control identifies the compound as a modulator of expression of the miRNA.

### III. Compositions

HSP90 inhibitor 17-AAG activity was shown to be enhanced by a series of miRNA. Therefore, up-regulating these specific microRNAs or providing analogous pharmaceutical compounds exogenously, should be effective for enhancement of HSP90 inhibitor in specific and any therapeutics in general.

In preferred embodiments, the miRNA formulations are administered to individuals with a cancer that has dysregulated HSP90 expression.

A. Nucleic Acid Sequences and Varients

Particularly favored embodiments of invention includes the following group of miRNAs as enhancers of HSP90 inhibitors: miR145

| | |
|---|---|
| (GUCCAGUUUUCCCAGGAAUCCCUU), | (SEQ ID NO: 1) |
| miR454-3p | |
| (UAGUGCAAUAUUGCUUAUAGGGUUU), | (SEQ ID NO: 2) |
| miR519a | |
| (AAAGUGCAUCCUUUUAGAGUGUUAC), | (SEQ ID NO: 3) |
| miR520c | |
| (AAAGUGCUUCCUUUUAGAGGGUU), | (SEQ ID NO: 4) |
| miR520d | |
| (AAAGUGCUUCUCUUUGGUGGGUU). | (SEQ ID NO: 5) |

Sequence variants of miRNA fall into one or more of three classes: substitutional, insertional or deletional variants. Insertions include 5′ and/or 3′ terminal fusions as well as intrasequence insertions of single or multiple residues, including at least 3, at least 5, at least 10, at least 15, at least 30, and at least 50 nucleotides. Insertions can also be introduced within the mature sequence. These, however, ordinarily will be smaller insertions than those at the 5′ or 3′ terminus, on the order of 1 to 4 residues.

Insertional sequence variants of miRNA are those in which one or more residues are introduced into a predetermined site in the target miRNA. Most commonly insertional variants are fusions of nucleic acids at the 5′ or 3′ terminus of the miRNA.

Deletion variants are characterized by the removal of one or more residues from the miRNA sequence. These variants ordinarily are prepared by site specific mutagenesis of nucleotides in the DNA encoding miRNA, thereby producing DNA encoding the variant, and thereafter expressing the DNA in recombinant cell culture. However, variant miRNA fragments may be conveniently prepared by in vitro synthesis. The variants typically exhibit the same qualitative biological activity as the naturally-occurring analogue, although variants also are selected in order to modify the characteristics of miRNA.

Substitutional variants are those in which, e.g., at least one to at least 3 nucleotides of the sequence has been removed and a different nucleotide inserted in its place. While the site for introducing a sequence variation is predetermined, the mutation per se need not be predetermined. For example, in order to optimize the performance of a mutation at a given site, random mutagenesis may be conducted at the target region and the expressed miRNA variants screened for the optimal combination of desired activity. Techniques for making substitution mutations at predetermined sites in DNA having a known sequence are well known.

Nucleotide substitutions are typically of single residues; insertions usually will be on the order of about from 1 to 10 residues; and deletions will range about from 1 to 30 residues. Deletions or insertions preferably are made in adjacent pairs; i.e. a deletion of 2 residues or insertion of 2 residues. Substitutions, deletion, insertions or any combination thereof may be combined to arrive at a final construct. Changes may be made to increase the activity of the miRNA, to increase its biological stability or half-life, and the like. All such modifications to the nucleotide sequences encoding such miRNA are encompassed.

The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence similarity percentages (e.g., BLASTN using default parameters) are generally available. Software for performing BLAST analyses is publicly available through the National Center for Biotechnology Information.

B. miRNAs that Enhance Therapeutic Potential of HSP90 Inhibitors

Naturally occurring microRNAs that regulate human oncogenes, pri-miRNA, pre-miRNA, mature miRNA or fragments of variants thereof that retain the biological activity of the mature miRNA and DNA encoding a pri-miRNA, pre-miRNA, mature miRNA, fragments or variants thereof, or regulatory elements of the miRNA, have been identified. The size of the miRNA is typically from 18 nucleotides to 170 nucleotides, although nucleotides of up to 2000 nucleotides can be utilized. In a preferred embodiment the size range of the pre-miRNA is between 70 to 170 nucleotides in length and the mature miRNA is between 21 and 25 nucleotides in length.

Synthetic miRNAs such as ds-miRNA and modified ds-miRNA allowing the incorporation of mature single stranded miRNA into the RISC complex can also be used. The size of the ds-miRNA range from 10-70 nucleotides in length.

The miRNA is selected from the group of miRNA shown to enhance the apoptotic activity of a HSP90 inhibitor—17-AAG. These include the following group of miRNAs miR145 (GUCCAGUUUUCCCAGGAAUCCCUU) (SEQ ID NO:1), miR454-3p (UAGUGCAAUAUUGCUUAUAGGGUUU) (SEQ ID NO:2), miR519a (AAAGUGCAUCCUUUUAGAGUGUUAC) (SEQ ID NO:3), miR520c (AAAGUGCUUCCUUUUAGAGGGUU) (SEQ ID NO:4), miR520d (AAAGUGCUUCUCUUUGGUGGGUU) (SEQ ID NO:5), as enhancer of HSP90 inhibitor or a mitotic inhibitor.

C. Nucleic Acids Techniques

General texts which describe molecular biological techniques include Sambrook, Molecular Cloning: a Laboratory Manual (2.sup.nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I. Theory and Nucleic Acid Preparation, P. Tijssen, ed. Elsevier, N.Y. (1993); Berger and Kimmel, Guide to Molecular Cloning Techniques Methods in Enzymology volume 152 Academic Press, Inc., San Diego, Calif. These texts describe mutagenesis, the use of vectors, promoters and many other relevant topics related to, e.g., the generation and expression of genes that encode let-7 or any other miRNA activity. Techniques for isolation, purification and manipulation of nucleic acids, genes, such as generating libraries, subcloning into expression vectors, labeling probes, and DNA hybridization are also described in the texts above and are well known to one of ordinary skill in the art.

The nucleic acids, whether miRNA, DNA, cDNA, or genomic DNA, or a variant thereof, may be isolated from a variety of sources or may be synthesized in vitro. Nucleic acids as described herein can be administered to or expressed in humans, transgenic animals, transformed cells, in a transformed cell lysate, or in a partially purified or a substantially pure form.

Nucleic acids are detected and quantified in accordance with any of a number of general means well known to those of skill in the art. These include, for example, analytical biochemical methods such as spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassays (RIAs), enzyme-linked immunosorbent assays (ELISAs), immuno-fluorescent assays, and the like, Southern analysis, Northern analysis, Dot-blot analysis, gel electrophoresis, RT-PCR, quantitative PCR, other nucleic acid or target or signal amplification methods, radiolabeling, scintillation counting, and affinity chromatography.

Various types of mutagenesis can be used, e.g., to modify a nucleic acid encoding a gene with miRNA activity. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, and mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, are also included in the present invention. In one embodiment, mutagenesis can be guided by known information of the naturally occurring molecule or altered or mutated naturally occurring molecule, e.g., sequence, sequence comparisons, physical properties, crystal structure or the like. Changes may be made to increase the activity of the miRNA, to increase its biological stability or half-life, and the like.

Comparative hybridization can be used to identify nucleic acids encoding genes with let-7 or other miRNA activity, including conservative variations of nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes part 1 chapter 2, “Overview of principles of hybridization and the strategy of nucleic acid probe assays,” (Elsevier, N.Y.), as well as in Ausubel, supra. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

The term “stringent hybridization conditions” is meant to refer to conditions under which a nucleic acid will hybridize to its target subsequence, typically in a complex mixture of nucleic acid, but to no other sequences. Stringent conditions are sequence-dependent and will be different in different circumstances. Longer sequences hybridize specifically at higher temperatures. Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5.times.SSC, and 1% SDS, incubating at 42° C. or, 5.times.SSC, 1% SDS, incubating at 65° C., with a wash in 0.2×SSC, and 0.1% SDS at 65° C.

Suitable nucleic acids for use in the methods described herein include, but are not limited to, pri-miRNA, pre-miRNA, ds miRNA, mature miRNA or fragments of variants thereof that retain the biological activity of the miRNA and DNA encoding a pri-miRNA, pre-miRNA, mature miRNA, fragments or variants thereof, or DNA encoding regulatory elements of the miRNA. In addition, DNA and PNA can replace RNA, provided the base pairing capabilities are maintained.

D. Vectors

In one embodiment the nucleic acid encoding a miRNA molecule is on a vector. These vectors include a sequence encoding a mature microRNA and in vivo expression elements. In a preferred embodiment, these vectors include a sequence encoding a pre-miRNA and in vivo expression elements such that the pre-miRNA is expressed and processed in vivo into a mature miRNA. In another embodiment, these vectors include a sequence encoding the pri-miRNA gene and in vivo expression elements. In this embodiment, the primary transcript is first processed to produce the stem-loop precursor miRNA molecule. The stem-loop precursor is then processed to produce the mature microRNA.

Vectors include, but are not limited to, plasmids, cosmids, phagemids, viruses, other vehicles derived from viral or bacterial sources that have been manipulated by the insertion or incorporation of the nucleic acid sequences for producing the microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One of skill in the art can readily employ other vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Genetically altered retroviral expression vectors have general utility for the high-efficiency transduction of nucleic acids in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991).

E. Promoters and Other Transcription/Expression Control Sequences

The “in vivo expression elements” are any regulatory nucleotide sequence, such as a promoter sequence or promoter-enhancer combination, which facilitates the efficient expression of the nucleic acid to produce the microRNA. The in vivo expression element may, for example, be a mammalian or viral promoter, such as a constitutive or inducible promoter or a tissue specific promoter. Examples of which are well known to one of ordinary skill in the art. Constitutive mammalian promoters include, but are not limited to, polymerase promoters as well as the promoters for the following genes: hypoxanthine phosphoribosyl transferase (HPTR), adenosine deaminase, pyruvate kinase, and beta.-actin. Exemplary viral promoters which function constitutively in eukaryotic cells include, but are not limited to, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. Inducible promoters are expressed in the presence of an inducing agent and include, but are not limited to, metal-inducible promoters and steroid-regulated promoters. For example, the metallothionein promoter is induced to promote transcription in the presence of certain metal ions. Other inducible promoters are known to those of ordinary skill in the art.

Examples of tissue-specific promoters include, but are not limited to, the promoter for creatine kinase, which has been used to direct expression in muscle and cardiac tissue and immunoglobulin heavy or light chain promoters for expression in B cells. Other tissue specific promoters include the human smooth muscle alpha-actin promoter.

Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol 7-alpha hydroylase (CYP-7) promoter, beta-galactosidase alpha-2,6 sialyltransferase promoter, insulin-like growth factor binding protein (IGFBP-1) promoter, aldolase B promoter, human transferrin promoter, and collagen type I promoter.

Exemplary tissue-specific expression elements for the prostate include but are not limited to the prostatic acid phosphatase (PAP) promoter, prostatic secretory protein of 94 (PSP 94) promoter, prostate specific antigen complex promoter, and human glandular kallikrein gene promoter (hgt-1).

Exemplary tissue-specific expression elements for gastric tissue include but are not limited to the human H+/K+-ATPase alpha subunit promoter.

Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter.

Exemplary tissue-specific expression elements for the endometrium include, but are not limited to, the uteroglobin promoter.

Exemplary tissue-specific expression elements for adrenal cells include, but are not limited to, cholesterol side-chain cleavage (SCC) promoter.

Exemplary tissue-specific expression elements for the general nervous system include, but are not limited to, gamma-gamma enolase (neuron-specific enolase, NSE) promoter.

Exemplary tissue-specific expression elements for the brain include, but are not limited to, the neurofilament heavy chain (NF-H) promoter.

Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′ transcriptional enhancer, and the human NK and T cell specific activation (NKG5) promoter.

Exemplary tissue-specific expression elements for the colon include, but are not limited to, pp 60c-src tyrosine kinase promoter, organ-specific neoantigens (OSNs) promoter, and colon specific antigen-P promoter.

Exemplary tissue-specific expression elements for breast cells include, but are not limited to, the human alpha-lactalbumin promoter.

Exemplary tissue-specific expression elements for the lung include, but are not limited to, the cystic fibrosis transmembrane conductance regulator (CFTR) gene promoter.

Other elements aiding specificity of expression in a tissue of interest can include secretion leader sequences, enhancers, nuclear localization signals, endosmolytic peptides, etc. Preferably, these elements are derived from the tissue of interest to aid specificity.

In general, the in vivo expression element shall include, as necessary, 5′ non-transcribing and 5′ non-translating sequences involved with the initiation of transcription. They optionally include enhancer sequences or upstream activator sequences.

F. Methods and Materials for Production of miRNA

The miRNA can be isolated from cells or tissues, recombinantly produced, or synthesized in vitro by a variety of techniques well known to one of ordinary skill in the art.

In one embodiment, miRNA is isolated from cells or tissues. Techniques for isolating miRNA from cells or tissues are well known to one of ordinary skill in the art. For example, miRNA can be isolated from total RNA using the mirVana miRNA isolation kit from Ambion, Inc. Another techniques utilizes the flashPAGE™ Fractionator System (Ambion, Inc.) for PAGE purification of small nucleic acids.

The miRNA can be obtained by preparing a recombinant version thereof (i.e., by using the techniques of genetic engineering to produce a recombinant nucleic acid which can then be isolated or purified by techniques well known to one of ordinary skill in the art). This embodiment involves growing a culture of host cells in a suitable culture medium, and purifying the miRNA from the cells or the culture in which the cells are grown. For example, the methods include a process for producing a miRNA in which a host cell containing a suitable expression vector that includes a nucleic acid encoding an miRNA is cultured under conditions that allow expression of the encoded miRNA. In a preferred embodiment the nucleic acid encodes let-7. The miRNA can be recovered from the culture, from the culture medium or from a lysate prepared from the host cells, and further purified. The host cell can be a higher eukaryotic host cell such as a mammalian cell, a lower eukaryotic host cell such as a yeast cell, or the host cell can be a prokaryotic cell such as a bacterial cell. Introduction of a vector containing the nucleic acid encoding the miRNA into the host cell can be effected by calcium phosphate transfection, DEAE, dextran mediated transfection, or electroporation (Davis, L. et al., Basic Methods in Molecular Biology (1986)).

Any host/vector system can be used to express one or more of the miRNAs. These include, but are not limited to, eukaryotic hosts such as HeLa cells and yeast, as well as prokaryotic host such as E. coli and B. subtilis. miRNA can be expressed in mammalian cells, yeast, bacteria, or other cells where the miRNA gene is under the control of an appropriate promoter. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989). In the preferred embodiment, the miRNA is expressed in mammalian cells. Examples of mammalian expression systems include C127, monkey COS cells, Chinese Hamster Ovary (CHO) cells, human kidney 293 cells, human epidermal A43 1 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Mammalian expression vectors will comprise an origin of replication, a suitable promoter, polyadenylation site, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Potentially suitable yeast strains include Saccharomyces cerevisiae, Schizosaccharomyces pombe, Kluyveromyces strains, Candida, or any yeast strain capable of expressing miRNA. Potentially suitable bacterial strains include Escherichia coli, Bacillus subtilis, Salmonella typhimurium, or any bacterial strain capable of expressing miRNA.

In a preferred embodiment, genomic DNA encoding miRNA selected from the group consisting of miR145 (GUCCAGUUUUCCCAGGAAUCCCUU) (SEQ ID NO: 1),

miR454-3p (UAGUGCAAUAUUGCUUAUAGGGUUU) (SEQ ID NO:2),
miR519a (AAAGUGCAUCCUUUUAGAGUGUUAC) (SEQ ID NO:3),
miR520c (AAAGUGCUUCCUUUUAGAGGGUU) (SEQ ID NO:4),
miR520d (AAAGUGCUUCUCUUUGGUGGGUU) (SEQ ID NO:5),
miR-425-3p (AUCGGGAAUGUCGUGUCCGCC) (SEQ ID NO:6),
miR-495 (AAACAAACAUGGUGCACUUCUUU) (SEQ ID NO:7),
miR-572 (GUCCGCUCGGCGGUGGCCCA) (SEQ ID NO:8), and/or
miR-661 (UGCCUGGGUCUCUGGCCUGCGCGU) (SEQ ID NO:9), is isolated, the genomic DNA is expressed in a mammalian expression system, RNA is purified and modified as necessary for administration to a patient. In a preferred embodiment the miRNA is in the form of a pre-miRNA, which can be modified as desired (i.e. for increased stability or cellular uptake).

Knowledge of DNA sequences of miRNA allows for modification of cells to permit or increase expression of an endogenous miRNA. Cells can be modified (e.g., by homologous recombination) to provide increased miRNA expression by replacing, in whole or in part, the naturally occurring promoter with all or part of a heterologous promoter so that the cells express the miRNA at higher levels. The heterologous promoter is inserted in such a manner that it is operatively linked to the desired miRNA encoding sequences. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also may be engineered to express an endogenous gene comprising the miRNA under the control of inducible regulatory elements, in which case the regulatory sequences of the endogenous gene may be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al.

The miRNA may be prepared by culturing transformed host cells under culture conditions suitable to express the miRNA. The resulting expressed miRNA may then be purified from such culture (i.e., from culture medium or cell extracts) using known purification processes, such as gel filtration and ion exchange chromatography. The purification of the miRNA may also include an affinity column containing agents which will bind to the protein; one or more column steps over such affinity resins as concanavalin A-agarose, Heparin-toyopearl™ or Cibacrom blue 3GA Sepharose™; one or more steps involving hydrophobic interaction chromatography using such resins as phenyl ether, butyl ether, or propyl ether; immunoaffinity chromatography, or complementary cDNA affinity chromatography.

The miRNA may also be expressed as a product of transgenic animals, which are characterized by somatic or germ cells containing a nucleotide sequence encoding the miRNA. A vector containing DNA encoding miRNA and appropriate regulatory elements can be inserted in the germ line of animals using homologous recombination (Capecchi, Science 244:1288-1292 (1989)), such that the express the miRNA. Transgenic animals, preferably non-human mammals, are produced using methods as described in U.S. Pat. No. 5,489,743 to Robinson, et al., and PCT Publication No. WO 94/28122 by Ontario Cancer Institute. miRNA can be isolated from cells or tissue isolated from transgenic animals as discussed above.

In a preferred embodiment, the miRNA can be obtained synthetically, for example, by chemically synthesizing a nucleic acid by any method of synthesis known to the skilled artisan. The synthesized miRNA can then be purified by any method known in the art. Methods for chemical synthesis of nucleic acids include, but are not limited to, in vitro chemical synthesis using phosphotriester, phosphate or phosphoramidite chemistry and solid phase techniques, or via deosynucleoside H-phosphonate intermediates (see U.S. Pat. No. 5,705,629 to Bhongle).

In some circumstances, for example, where increased nuclease stability is desired, nucleic acids having nucleic acid analogs and/or modified internucleoside linkages may be preferred. Nucleic acids containing modified internucleoside linkages may also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH.sub.2-S—CH.sub.2), dimethylene-sulfoxide (—CH.sub.2-SO—CH.sub.2), dimethylene-sulfone (—CH.sub.2-SO.sub.2-CH.sub.2), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., U.S. Pat. No. 5,714,606 to Acevedo, et al., U.S. Pat. No. 5,378,825 to Cook, et al., U.S. Pat. Nos. 5,672,697 and 5,466,786 to Buhr, et al., U.S. Pat. No. 5,777,092 to Cook, et al., U.S. Pat. No. 5,602,240 to De Mesmaeker, et al., U.S. Pat. No. 5,610,289 to Cook, et al. and U.S. Pat. No. 5,858,988 to Wang, also describe nucleic acid analogs for enhanced nuclease stability and cellular uptake.

### IV. Formulations

The compositions are administered to a patient in need of treatment or prophylaxis of at least one symptom or manifestation (since disease can occur/progress in the absence of symptoms) of cancer/proliferative disease. Aberrant expression of oncogenes is a hallmark of cancer. In preferred embodiments, the compositions are administered in an effective amount to enhance the therapeutic activity of hsp90 inhibitor 17-AAG.

Methods for treatment or prevention of at least one symptom or manifestation of cancer are also described consisting of administration of an effective amount of a composition containing a nucleic acid molecule to alleviate at least one symptom or decrease at least one manifestation. In a preferred embodiment, the cancer is lung cancer. The compositions described herein can be administered in effective dosages alone or in combination with adjuvant cancer therapy such as surgery, chemotherapy, radiotherapy, thermotherapy, immunotherapy, hormone therapy and laser therapy, to provide a beneficial effect, e.g. reduce tumor size, reduce cell proliferation of the tumor, inhibit angiogenesis, inhibit metastasis, or otherwise improve at least one symptom or manifestation of the disease.

The nucleic acids described above are preferably employed for therapeutic uses in combination with a suitable pharmaceutical carrier. Such compositions comprise an effective amount of the compound, and a pharmaceutically acceptable carrier or excipient. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids some of which are described herein.

It is understood by one of ordinary skill in the art that nucleic acids administered in vivo are taken up and distributed to cells and tissues (Huang, et al., FEBS Lett. 558(1-3):69-73 (2004)). For example, Nyce et al. have shown that antisense oligodeoxynucleotides (ODNs) when inhaled bind to endogenous surfactant (a lipid produced by lung cells) and are taken up by lung cells without a need for additional carrier lipids (Nyce and Metzger, Nature, 385:721-725 (1997). Small nucleic acids are readily taken up into T24 bladder carcinoma tissue culture cells (Ma, et al., Antisense Nucleic Acid Drug Dev. 8:415-426 (1998). siRNAs have been used for therapeutic silencing of an endogenous genes by systemic administration (Soutschek, et al., Nature 432, 173-178 (2004)).

The nucleic acids described above may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The nucleic acids may also be encapsulated in suitable biocompatible microcapsules, microparticles or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example in Sambrook et al., 1989, Molecular Cloning: A Laboratory Manual, Cold Spring Harbor Laboratory, New York; and Ausubel et al., 1994, Current Protocols in Molecular Biology, John Wiley & Sons, New York. Such nucleic acid delivery systems comprise the desired nucleic acid, by way of example and not by limitation, in either “naked” form as a “naked” nucleic acid (e.g., in saline or D5), or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. By way of example, and not by limitation, the nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, thickeners and the like can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, with an added preservative. The compositions may take such forms as.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The nucleic acids alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. For administration by inhalation, the nucleic acids are conveniently delivered in the form of an aerosol spray presentation from pressurized packs or a nebulizer, with the use of a suitable propellant.

In some embodiments, the nucleic acids described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the nucleic acids are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett. 14(19):4975-4977 (2004)) and in vivo (Soutschek, et al., Nature 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol. 59(11):1407-1416 (2000)). Other groups that can be attached or conjugated to the nucleic acids described above to increase cellular uptake, include, but are not limited to, acridinederivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also described methods for enhanced delivery of nucleic acids molecules.

These pharmaceutical formulations may be manufactured in a manner that is itself known, e.g., by means of conventional mixing, dissolving, granulating, levigating, emulsifying, encapsulating, entrapping or lyophilizing processes.

The formulations described herein of the nucleic acids embrace fusions of the nucleic acids or modifications of the nucleic acids, wherein the nucleic acid is fused to another moiety or moieties, e.g., targeting moiety or another therapeutic agent. Such analogs may exhibit improved properties such as activity and/or stability. Examples of moieties which may be linked or unlinked to the nucleic acid include, for example, targeting moieties which provide for the delivery of nucleic acid to specific cells, e.g., antibodies to pancreatic cells, immune cells, lung cells or any other preferred cell type, as well as receptor and ligands expressed on the preferred cell type. Preferably, the moieties target cancer or tumor cells. For example, since cancer cells have increased consumption of glucose, the nucleic acids can be linked to glucose molecules. Monoclonal humanized antibodies that target cancer or tumor cells are preferred moieties and can be linked or unlinked to the nucleic acids. In the case of cancer therapeutics, the target antigen is typically a protein that is unique and/or essential to the tumor cells (e.g., the receptor protein HER-2).

### V. Methods of Treatment

A. Method of Administration

In general, methods of administering nucleic acids are well known in the art. In particular, the routes of administration already in use for nucleic acid therapeutics, along with formulations in current use, provide preferred routes of administration and formulation for the nucleic acids described above.

Nucleic acid compositions can be administered by a number of routes including, but not limited to: oral, intravenous, intraperitoneal, intramuscular, transdermal, subcutaneous, topical, sublingual, or rectal means. Nucleic acids can also be administered via liposomes. Such administration routes and appropriate formulations are generally known to those of skill in the art.

Administration of the formulations described herein may be accomplished by any acceptable method which allows the miRNA or nucleic acid encoding the miRNA to reach its target. The particular mode selected will depend of course, upon factors such as the particular formulation, the severity of the state of the subject being treated, and the dosage required for therapeutic efficacy. As generally used herein, an “effective amount” of a nucleic acids is that amount which is able to treat one or more symptoms of cancer or related disease, reverse the progression of one or more symptoms of cancer or related disease, halt the progression of one or more symptoms of cancer or related disease, or prevent the occurrence of one or more symptoms of cancer or related disease in a subject to whom the formulation is administered, as compared to a matched subject not receiving the compound or therapeutic agent. The actual effective amounts of drug can vary according to the specific drug or combination thereof being utilized, the particular composition formulated, the mode of administration, and the age, weight, condition of the patient, and severity of the symptoms or condition being treated.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. The composition can be injected intradermally for treatment or prevention of cancer, for example. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

Preferably, the agent and/or nucleic acid delivery system are provided in a manner which enables tissue-specific uptake of the agent and/or nucleic acid delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the miRNA over a certain time period, for example, hours, days, weeks, months or years. This may be accomplished, for example, by repeated administrations of a formulation or by a sustained or controlled release delivery system in which the miRNA is delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include, but are not limited to, time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the miRNA is contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the miRNA. In addition, a pump-based hardware delivery system may be used to deliver one or more embodiments.

Examples of systems in which release occurs in bursts includes, e.g., systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be e.g., in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the miRNA employed and the condition of the patient, as well as the body weight or surface area of the patient to be treated. The size of the dose is also determined by the existence, nature, and extent of any adverse side-effects that accompany the administration of a particular vector, formulation, or the like in a particular patient.

Therapeutic compositions comprising one or more nucleic acids are optionally tested in one or more appropriate in vitro and/or in vivo animal models of disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of the nucleic acids at various concentrations, e.g., as applied to the mass and overall health of the patient. Administration can be accomplished via single or divided doses.

In vitro models can be used to determine the effective doses of the nucleic acids as a potential cancer treatment. Suitable in vitro models include, but are not limited to, proliferation assays of cultured tumor cells, growth of cultured tumor cells in soft agar (see Freshney, (1987) Culture of Animal Cells: A Manual of Basic Technique, Wily-Liss, New York, N.Y. Ch 18 and Ch 21), tumor systems in nude mice as described in Giovanella et al., J. Natl. Can. Inst., 52: 921-30 (1974), mobility and invasive potential of tumor cells in Boyden Chamber assays as described in Pilkington et al., Anticancer Res., 17: 4107-9 (1997), and angiogenesis assays such as induction of vascularization of the chick chorioallantoic membrane or induction of vascular endothelial cell migration as described in Ribatta et al., Intl. J. Dev. Biol., 40: 1189-97 (1999) and Li et al., Clin. Exp. Metastasis, 17:423-9 (1999), respectively. Suitable tumor cells lines are available, e.g. from American Type Tissue Culture Collection catalogs.

In vivo models are the preferred models to determine the effective doses of nucleic acids described above as potential cancer treatments. Suitable in vivo models include, but are not limited to, mice that carry a mutation in the KRAS oncogene (Lox-Stop-Lox K-Ras.sup.G12D mutants, Kras2.sup.tm4TYj) available from the National Cancer Institute (NCI) Frederick Mouse Repository. Other mouse models known in the art and that are available include but are not limited to models for gastrointestinal cancer, hematopoietic cancer, lung cancer, mammary gland cancer, nervous system cancer, ovarian cancer, prostate cancer, skin cancer, cervical cancer, oral cancer, and sarcoma cancer (see http://emice.nci.nih.gov/mouse_models/).

In determining the effective amount of the miRNA to be administered in the treatment or prophylaxis of disease the physician evaluates circulating plasma levels, formulation toxicities, and progression of the disease.

The dose administered to a 70 kilogram patient is typically in the range equivalent to dosages of currently-used therapeutic antisense oligonucleotides such as Vitravene® (fomivirsen sodium injection) which is approved by the FDA for treatment of cytomegaloviral RNA, adjusted for the altered activity or serum half-life of the relevant composition.

The formulations described herein can supplement treatment conditions by any known conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, the nucleic acids can also be administered in therapeutically effective amounts as a portion of an anti-cancer cocktail. An anti-cancer cocktail is a mixture of the oligonucleotide or modulator with one or more anti-cancer drugs in addition to a pharmaceutically acceptable carrier for delivery. The use of anti-cancer cocktails as a cancer treatment is routine. Anti-cancer treatments include: The composition of any of claims 29-40, wherein the therapeutic agent is selected is a radionuclide, cancer chemotherapeutic agent, targeted anticancer agent, DNA intercalating/damaging agent, cell cycle check point inhibitor, anti-metabolites, HSP inhibitor, antibiotic, kinase inhibitor, radionuclide, biologically active polypeptide, antibody, lectin, toxin, hormone, matrix metalloproteinase inhibitors, angiostatic steroid or combinations thereof. Further, that are well known in the art and can be used as a treatment in combination with the nucleic acids described herein include, but are not limited to: ¹³¹I, 9⁰Y, ¹¹¹In, ²¹¹At, ³²P, genistein, adriamycin, ansamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin, sirolimus, mitomycin, mitotane, mitoxantrone, nitrosurea, pamidronate, pentostatin, plicamycin, procarbazine, rituximab, streptozocin, teniposide, thioguanine, thiotepa, taxanes, vinblastine, vincristine, vinorelbine, taxol, combretastatins, discodernolides, transplatinum, bleomycin, hormones, tamoxifen, diethylstilbestrol, axitinib, avastin, marimastat, bevacizumab, carboxyamidotriazole, TNP-470, CM101, IFN-α, IL-12, platelet factor-4, suramin, SU5416, thrombospondin, VEGFR antagonists, cartilage-derived angiogenesis inhibitory factor, angiostatin, endostati, 2-methoxyestradiol, tecogalan, thrombospondin, prolactin, αvβ3 inhibitors, tecogalan, BAY 12-9566, AG3340, CGS27023A, COL-3, vitaxin, ZD0101, TNP-40, thalidomide, squalamine, IM862, PTK787, fumagillin, analogues of fumagillin, BB-94, BB-2516 linomid, 17-AAG, oxaliplatin, paclitaxel and combinations thereof.

### VI. Diseases Treated

Neurodegenerative diseases such as Alzheimer's, Pakinson's, ALS, and spinal and bulbar muscular dystrophy.

Proliferative disease is selected from the group consisting of hypertrophic scars and keloids, proliferative diabetic retinopathy, rheumatoid arthritis, arteriovenous malformations, atherosclerotic plaques, delayed wound healing, hemophilic joints, nonunion fractures, Osler-Weber syndrome, psoriasis, pyogenic granuloma, scleroderma, tracoma, menorrhagia, vascular adhesions and restenosis.

Cancer treatments promote tumor regression by inhibiting tumor cell proliferation, inhibiting angiogenesis (growth of new blood vessels that is necessary to support tumor growth) and/or prohibiting metastasis by reducing tumor cell motility or invasiveness. Therapeutic formulations described herein may be effective in adult and pediatric oncology including in solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma. Therapeutic formulations can be administered in therapeutically effective dosages alone or in combination with adjuvant cancer therapy such as surgery, chemotherapy, radiotherapy, thermotherapy, immunotherapy, hormone therapy and laser therapy, to provide a beneficial effect, e.g. reducing tumor size, slowing rate of tumor growth, reducing cell proliferation of the tumor, promoting cancer cell death, inhibiting angiongenesis, inhibiting metastasis, or otherwise improving overall clinical condition, without necessarily eradicating the cancer.

Cancers include, but are not limited to, biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor. In a preferred embodiment, the formulations are administered for treatment or prevention of lung cancer.

Accordingly, the inventive methods and compositions disclosed herein can be used by human patient undergoing one or more cancer therapies selected from the group consisting of surgery, chemotherapy, radiotherapy, thermotherapy, immunotherapy, hormone therapy and laser therapy. further the methods and compositions of the invention allow for use in a human patient undergoing one or more antiproliferative therapies consisting of surgery, chemotherapy, radiotherapy, thermotherapy, immunotherapy, hormone therapy, laser therapy, or stenting.

In addition, therapeutic nucleic acids may be used for prophylactic treatment of cancer. There are hereditary conditions and/or environmental situations (e.g. exposure to carcinogens) known in the art that predispose an individual to developing cancers. Under these circumstances, it may be beneficial to treat these individuals with therapeutically effective doses of the nucleic acids to reduce the risk of developing cancers. In one embodiment, a nucleic acid in a suitable formulation may be administered to a subject who has a family history of cancer, or to a subject who has a genetic predisposition for cancer. In other embodiments, the nucleic acid in a suitable formulation is administered to a subject who has reached a particular age, or to a subject more likely to get cancer. In yet other embodiments, the nucleic acid in a suitable formulation is administered to subjects who exhibit symptoms of cancer (e.g., early or advanced). In still other embodiments, the nucleic acid in a suitable formulation may be administered to a subject as a preventive measure. In some embodiments, the nucleic acid in a suitable formulation may be administered to a subject based on demographics or epidemiological studies, or to a subject in a particular field or career.

The following examples further illustrate the invention but, of course, should not be construed as in any way limiting its scope.

### Example 1

This Example demonstrates the apoptotic activity of the HSP90 inhibitor 17-AAG.

The Apo-ONE® Homogeneous Caspase-3/7 Assay (Promega, Madison, Wis.) uses a proprietary lysis/activity buffer, in conjunction with the (Z-DEVD)2-Rhodamine 110 substrate, enables a simple “add-mix-read” format for the detection of caspase-3 and -7 in adherent, suspension, and primary culture cells, or in purified caspase preparations. The assay uses a rhodamine 110-based substrate allows for exquisite sensitivity previously unobtainable with conventional colorimetric or fluorometric assays.

Specifically, 100 μl of Apo-ONE® Caspase-3/7 Reagent was added to each well of a white or black 96-well plate containing 100 μl of blank, control or cells in culture. The plate was covered with a plate sealer for incubating for extended periods (>4 hours). In order to perform this assay in a 384-well plate, a 1:1 volume ratio of Apo-ONE® Caspase-3/7 Reagent to sample was used. The contents of wells were mixed using a plate shaker at 300-500 rpm from 30 seconds and incubated at room temperature for 6 hours. The fluorescence of each well was determined at 485/538 nm (a measure of apoptosis, with the higher ratio indicating more apoptosis).

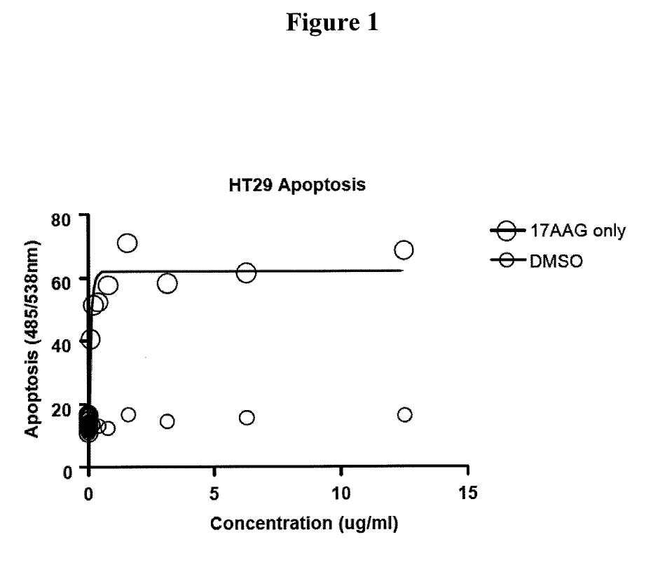

The apoptotic activity of 17-AAG (17-(Allylamino)-17-demethoxygeldanamycin) was determined in the colon cancer line HT29 using an assay for Caspase-3/7 activity (Apo-ONE kit, Promega). 17-AAG-treated HT29 cells were compared with DMSO-treated control (FIG. 1). HT29 Cells are treated with 17-AAG of various concentrations at 37° C. for 72 hours. 17-AAG induced the apoptosis with EC50 of 0.07 μg/ml or 0.12 μM. At higher concentration of 17-AAG, cell death occurred via non-apoptotic route and there was an artifactual reduction in apoptotic activity.

### Example 2

This Example demonstrates the suppression of Her2 expression by the HSP90 inhibitor 17-AAG.

Ten thousand BT474 cells per well were seeded into a microtiter plate and grown for 48 hours. After this preincubation, the BT474 cells treated with various concentrations of 17-AAG and its analogs for 24-hours. At the end of this incubation the media was removed from each well, each well was washed twice with ice-cold Tris buffer saline (containing 0.1% Tween 20) and the cells were fixed with Methanol (ice-cold) at 40° C. for 10 minutes. The fixed BT474 cells were immunostained with anti-Her2 antibody. The presence of Her2 protein was determined by measurement of the absorbance at 405 nm in the plate reader.

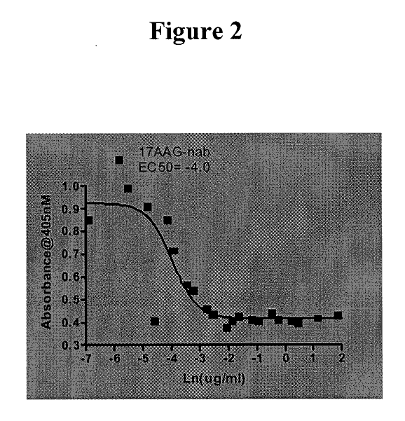

As shown in FIG. 2, the IC50 of 17-AAG for Her2 suppression assay was closed to 32 nM. This result suggested that Her2 protein expression is strongly suppressed by 17-AAG.

### Example 3

This Example demonstrates that there is internalization and degradation of Her2 following inhibition of HSP90 by 17-AAG.

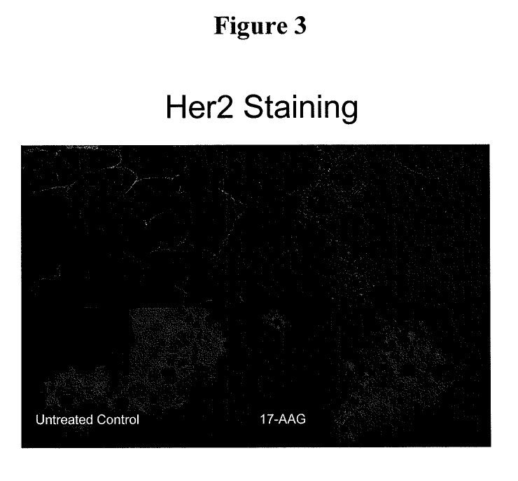

17-AAG treated BT474 cells were examined by the confocal images system. BT474 cells were seeded on the slide and treated at the concentration of IC50 for 24 hours. 17AAG-treated and control BT474 cells were fixed with Methanol, stained with Her2 antibody and analyzed by the confocal image. As shown in FIG. 3, the Her2 protein expression was eliminated from its cell surface sub-localization to cytoplasm after 17-AAG treatment.

### Example 4

This Example demonstrates that combination therapy with 17-AAG and chemotherapy upregulates Hsp70. Thus, HSP90 therapy is limited by the compensatory increase in HSP70.

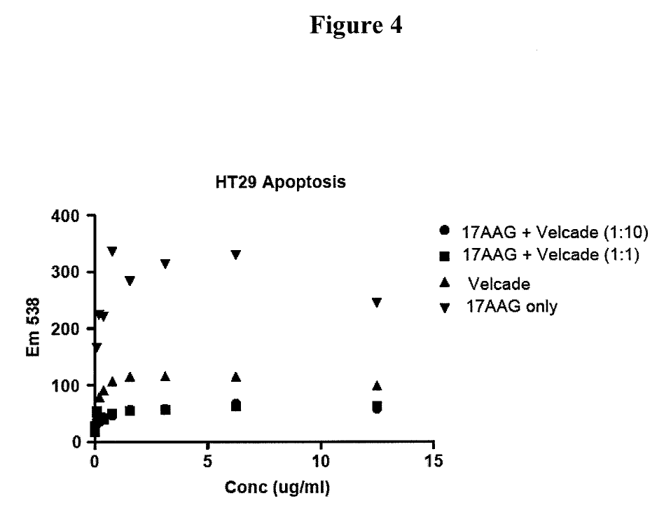

A panel of anticancer chemotherapeutical agents was tested for the enhancement of apoptotic activity with 17-AAG (using the same apoptotic assay system in Example 1), FIG. 4. HT29 cells were treated with various concentration of 17-AGG in the presence or absence of anticancer agents including known HSP70 promoter inducers. Three days after the drug treatment, the apoptotic activity was measured by the Apo-ONE kit (see above). When combined with the anticancer agents, including HSP70 inducers (such as Cisplatin, Etoposide, Doxorubicin, Velcade, Dimethylenastron). The apoptotic activity of 17-AAG was strongly inhibited in the presence of Velcade which is known to induce HSP70 expression (see Lauricella M. et al. Apoptosis. 2006 April; 11(4):607-25). Some inhibition was also observed for other inducers of HSP70.

### Example 5

This Example demonstrates the enhancement of 17-AAG activity by miRNAs.

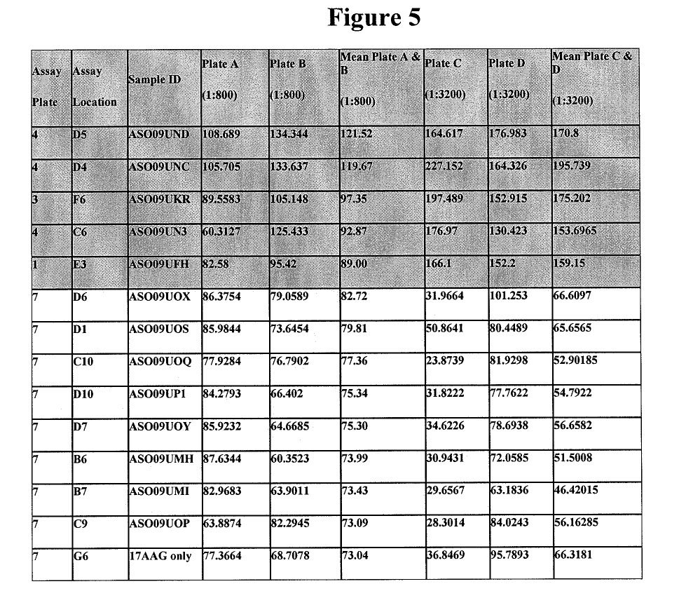

In order to observe an enhancement of activity in 17-AAG, a miRNA library of 470 Pre-miRNA precursors have been transfected (Ambion, siPORT NeoFx transfection agent) in to the HT29 colon cancer line and followed by 17-AAG treatment. Synthetic ds-miRNAs from the miRNA library were transfected into HT29 cells and incubated for 48 hours at 37° C. Various concentrations of 17-AAG were added to miRNA transfected HT29 cells and incubated for another 48 hours. 100 uL of media was aspirated from each well. Apo-One caspase reagent was prepared by diluting the substrate in buffer in a 1:100 concentration. 100 uL of the reagent was added to each well. Plates were incubated at room temperature for 6 hours. The apoptotic activity was measured by the Fluoroskan plate reader. Molecules that synergized with 17-AAG by exhibiting an apoptotic reading than 17-AAG alone. Molecules that synergized at both 1:800 and 1:3200 dilutions of 17-AAG were miR-145, miR-454-3p, miR519a, miR-520c, and miR-520d (SEQ ID NOS:1-5), (FIG. 5, dark background, respectively).

### Example 6

This Example confirms some of the results from Example 5.

The same procedure used in Example 5 was used to transfect the 5 miRNAs found identified in Example 5. However, 17-AAG was added in a 1:800, 1:3200 and 1:8000 to further define its optimal concentration for apoptotic activity. The cells were incubated for 48 hours, and developed using the same procedure as described in Example 5.

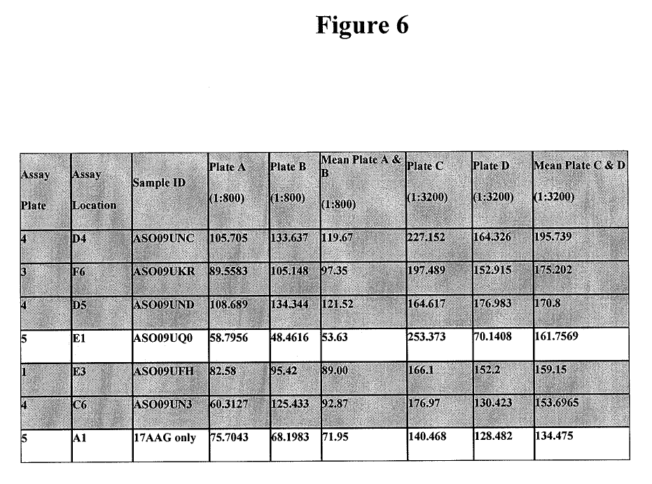

As shown in FIGS. 6 (dark) and 7, all 5 miRNAs enhanced the activity of 17-AAG while none had activity alone. “DMSO” represents cells transfected with the miRNAs but not treated with 17-AAG.

This experiment was repeated using varying miRNA concentrations (6, 3, 1.5 and 0.75 pmol) for the transfection to optimize the working dose for miRNA. A 1:3200 concentration of 17-AAG in growth medium was used.

### Example 7

This Example demonstrates the similarity between the miRNAs found to enhance 17-AAG induced killing.

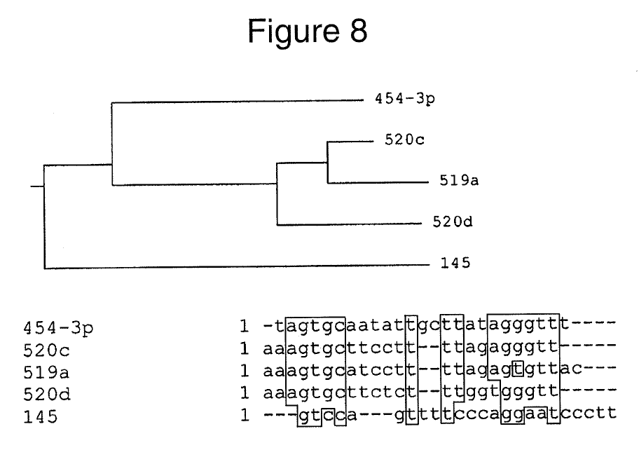

The sequences of the identified miRNAs were compared shown to share stretches of residues that are conserved. One miRNA, hsa-mir-145, shared less sequence homology, while the others hsa-mir-519a, hsa-mir-520c, hsa-mir-520d, and hsa-454-3p (SEQ ID NOS: 3-6 respectively), showed extensive homology to one another (FIG. 8). Thus, there are two classes of targets controlling by these miRNA.

### Example 8

This Example demonstrates that 17-AAG synergy increased with increasing amount of miRNA used.

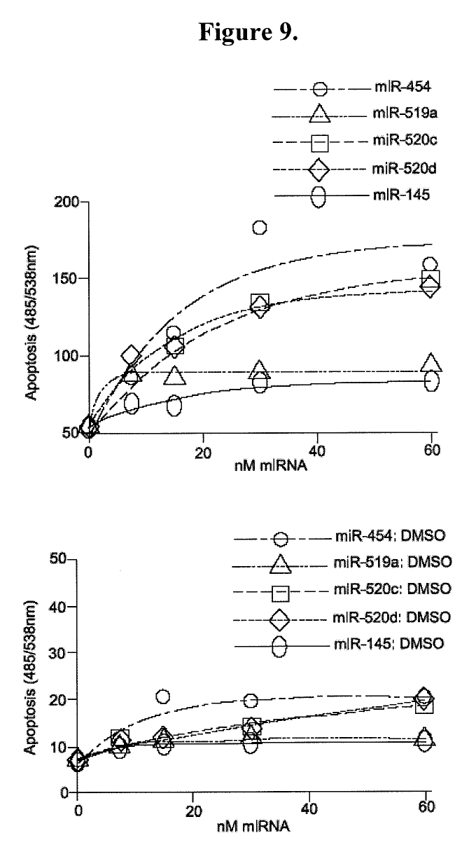

To confirm the activity shown in Example 6, the experiment was repeated with constant amount of 17-AAG (3.125 ug/ml) and increasing amount of miRNAs (60, 30, 15, 7.5 nM) used for transfection. Synergy with 17-AAG exhibited increasing activity with increasing amount of miRNA used (see FIG. 9 A). No apoptotic activity was observed for miRNA alone (see FIG. 9 B). There were two distinct classes of miRNAs according to activity (one class with high activity: miR454 (SEQ ID NO:2), miR-520c (SEQ ID NO:4) and miR-520d (SEQ ID NO:5); one class with low activity: miR-145 (SEQ ID NO:1) and miR-519a (SEQ ID NO:3)).

### Example 9

This Example presents the protein expression profile of HT29 tumor cells treated with the miRNAs.

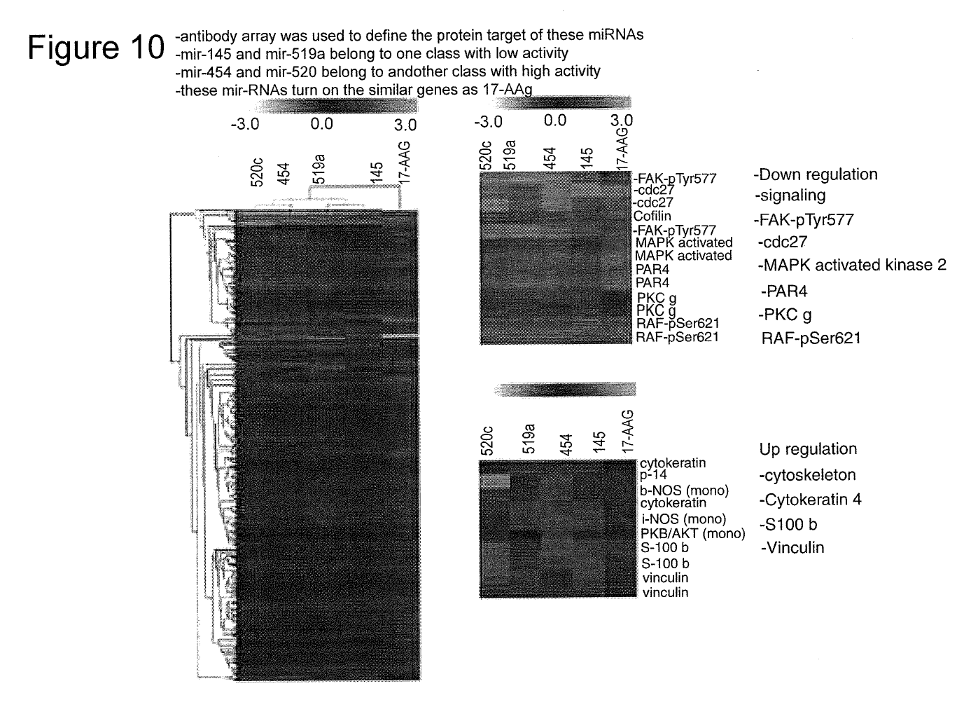

To determine the global protein expression profile of cell treated with these miRNAs, an antibody array containing 224 human antibodies to key cellular proteins with a special emphasis on cell signaling proteins was used. (FIG. 10) The antibodies are spotted in duplicate on a nitrocellulose-coated glass slide and can detect protein levels as low as a few nanograms per ml. Cell lysate from untreated HT29 cells was labeled with Cy5. Cell lysates from HT29 cells treated one of the four miRNAs, (SEQ ID NOS: 1-4), or (SEQ ID NOS: 1, 2, 3, OR 4). The antibody array was reacted to an equal mix of Cy3/Cy5 (treated/untreated) lysates, wash, and scanned. The log normalized ratios of treated/untreated (treated separately with 17-AAG, mir-145, mir-454, mir519a, or mir-520c (SEQ ID NOs:2-4, respectively)) were subjected to cluster analysis to determine proteins that are modulated similarly by the 4 miRNAs. As shown in FIG. 10, the protein profiles of all five treated samples were similar—indicating that these miRNAs are acting on the same pathway and this pathway is also the same as that acted on by 17-AAG, producing synergy. Furthermore, cluster analysis indicated that 519a and 145 belong to one class and 520c and 519a belong to another class—consistent with conclusion arrived at using activity assay. Genes which are down regulated by these miRNAs in common with 17-AAG are signaling molecules and include: FAK-pTyr577, cdc27, MAPK activated kinase 2, PAR4, PKC gamma, and RAF-pSer621. Genes which are up regulated by these miRNAs in common with 17-AAG are cytoskeletal elements and include: cytokeratin 4, S100 b, and vinculin.

In addition—hsa-mir-520c (SEQ ID NO:4) has been shown to modulated CD44 translation (Huang Q et al., 2008). The microRNAs mir-373 (GAAGUGCUUCGAU UUUGGGGUGU) (SEQ ID NO:39) and mir-520c (SEQ ID NO:4) promote tumour invasion and metastasis. Nature Cell Biology 10:202-10. This would make CD44, CDC27, MAPK activated kinase 2, PAR4, PKC gamma as potential targets for these miRNAs,

### Example 10

This Example shows the stringent sequence requirement of these miRNAs not anticipated from the current understanding of miRNA.

As the miRNA database been constantly updated and changes, there exist several reported versions of the five miRNAs shown to enhance 17-AAG apoptotic activity. The active forms of these miRNAs were inactivated when as few a single residue was deleted from the 3′ end as shown in the following table:

| | |
|---|---|
| Active | Inactive |

| | | |
|---|---|---|
| Hsa-mir-145 | GUCCAGUUUUCCCAGGAAUCCCUU | GUCCAGUUUUCCCAGGAAUCCCU |
| (SEQ ID NO: 1) | | |
| Hsa-mir-519a | AAAGUGCAUCCUUUUAGAGUGUUAC | AAAGUGCAUCCUUUUAGAGUGU |
| (SEQ ID NO: 3) | | |
| Hsa-mir-454-3p | UAGUGCAAUAUUGCUUAUAGGGUUU | UAGUGCAAUAUUGCUUAUAGGGU |
| (SEQ ID NO: 2) | | |
| Hsa-mir-520c | AAAGUGCUUCCUUUUAGAGGGUU | AAAGUGCUUCCUUUUAGAGGGU |
| (SEQ ID NO: 4) | | |

### Example 11

This Example demonstrates that specific miRNAs can enhance the apoptotic effect of specific therapeutic agents.

A transfection medium was made up by diluting siPORT NeoFx (0.3 μl/well) in OPTI-MEM (16.7 ul/well) in the wells of a microtiter plate and this medium was incubated for 10 minutes at room temperature. A pre-miRNA library was diluted to 1 μM and 27 μl of miRNA was added to each well (30 nM). This mixture was incubated at room temperature for another 10 minutes. HT29 cells were then added at 30,000 cells/well and the plates were incubated 48 hours 37° C.

For treatment with candidate therapeutic agents, the media was aspirated from each well and 200 μl/well of 15.6 μg/ml oxaliplatin, 7.8 μg/ml paclitaxel or negative control solution was added. The plates were incubated for 48 hours 37° C. The Apo-ONE® Homogeneous Caspase-3/7 Assay (Promega, Madison, Wis., USA) was performed on the treated cells. The reactions were performed in duplicate.

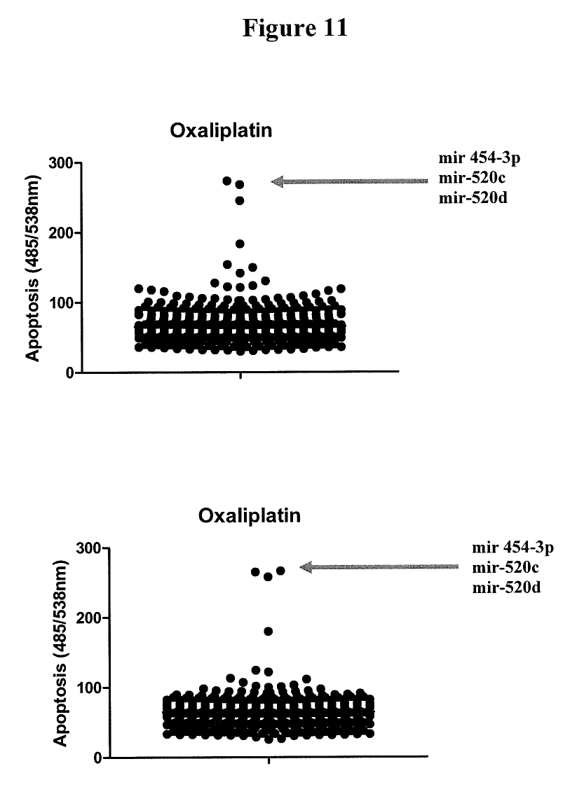

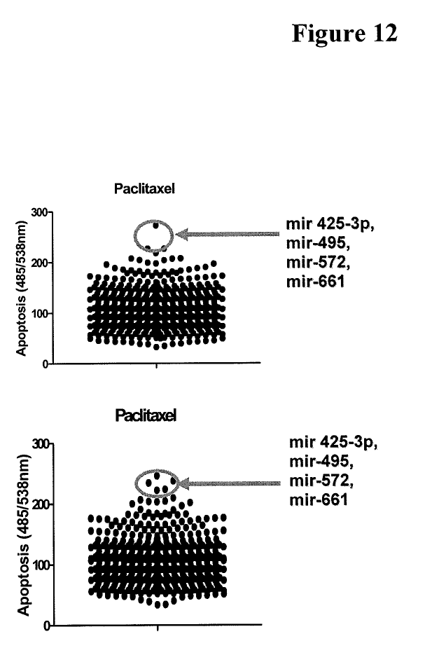

The results for the oxaliplatin are shown in FIG. 11, at 15.6 μg/ml oxaliplatin, highest level of apoptosis was obtained in oxaliplatin treated cells expressing the exogenous miRNAs mir 454-3p, mir 520c, and mir 520d. (SEQ ID NOS: 2, 3, and 4, respectively) (Notably, these miRNA also enhanced 17-AAG's apoptotic activity.) For paclitaxel, the expression of mir 425-3p, mir 495. mir 572, and mir 661. (SEQ ID NOS: 6-9, respectively) led to the highest levels of apoptosis (FIG. 12).

Thus, specific miRNAs can be used to enhance the activity of specific agents and the assay in this Example can be adapted to identify such miRNAs.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for enhancing the activity of a therapeutic agent in an organism afflicted with cancer, neurodegenerative diseases, restenosis or proliferative cellular diseases comprising administering an effective amount of a composition comprising an miRNA before, during or after administering the therapeutic agent.

2. The method of claim 1, wherein the miRNA is selected from the group consisting of a pri-miRNA, pre-miRNA, mature miRNA, ds miRNA and fragments or variants thereof.

3. The method of claim 2, wherein the miRNA is encoded by an isolated nucleic acid.

4. The method of claim 3, wherein the isolated nucleic acid is integrated into a vector.

5. The method of claim 4, wherein the vector is selected from the group consisting of a plasmid, cosmid, phagemid, virus, and artificial chromosome.

6. The method of claim 4, wherein the vector further comprises one or more in vivo expression control elements.

7. The method of claim 6, wherein the one or more in vivo expression element is selected from the group consisting of a promoter, enhancer, RNA splice sites, and combinations thereof.

8. The method of claim 7, wherein the isolated nucleic acid is transfected into the cells of the organism.

9. The method of claim 1, wherein the miRNA is a naked synthetic RNA.

10. The method of claim 1, wherein the miRNA is a chemically modified synthetic RNA.

11. The method of claim 10, wherein the synthetic RNA is modified with a chemical moiety selected from the group consisting of phosphorothioate, boranophosphate, 2′-O-methyl, 2′-fluoro, PEG, terminal inverted-dT base, and combinations thereof.

12. The method of claim 1, wherein the miRNA is administered in a liposome, polymer-based nanoparticle, cholesterol conjugate, cyclodextran complex, polyethylenimine polymer or a protein complex.

13. The method of claim 1, wherein the miRNA is administered directly to the diseased tissue in the organism, intravenously, subcutaneously, intramuscularly, nasally, intraperitonealy, vaginally, anally, orally, intraocularly or intrathecally.

14. The method of claim 1, wherein the miRNA is from 18 nucleotides to 170 nucleotides in length.

15. The method of claim 14, wherein the miRNA is from 18 to 25 nucleotides in length.

16. The method of claim 15, wherein the therapeutic agent is selected from the group consisting of radionuclides, chemotherapeutic agents, targeted anticancer agents, DNA interacalating/damaging agents, cell cycle check point inhibitors, anti-metabolites, heat shock protein inhibitors, kinase inhibitors, and combinations thereof.

17. The method of claim 1, wherein the therapeutic agent is selected from the group consisting of genistein, ¹³¹I, ⁹⁰Y, ¹¹¹In, ²¹¹At, ³²P, adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin, sirolimus, mitomycin, mitotane, mitoxantrone, nitrosurea, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, transplatinum, bleomycin, hormones, tamoxifen, diethylstilbestrol, biologically active polypeptides, antibodies, lectins, toxins, Axitinib, Avastin, marimastat, bevacizumab, carboxyamidotriazole, TNP-470, CM101, IFN-α, IL-12, platelet factor-4, suramin, SU5416, thrombospondin, VEGFR antagonists, angiostatic steroids, cartilage-derived angiogenesis inhibitory factor, matrix metalloproteinase inhibitors, angiostatin, endostati, 2-methoxyestradiol, tecogalan, thrombospondin, prolactin, αvβ3 inhibitors, tecogalan, BAY 12-9566, AG3340, CGS27023A, COL-3, vitaxin, ZD0101, TNP-40, thalidomide, squalamine, IM862, PTK787, fumagillin, analogues of fumagillin, BB-94, BB-2516 linomid, 17-AAG, oxaliplatin, paclitaxel and combinations thereof.

18. The method of claim 17, wherein the therapeutic agent is 17-AAG, oxaliplatin, paclitaxel or a combination thereof.

19. The method of claim 18, wherein
(a) the cancer is selected from the group consisting of circinoma in situ, atypical hyperplasia, carcinoma, sarcoma, carcinosarcoma, lung cancer, pancreatic cancer, skin cancer, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer,
(b) the restenosis is selected from the group consisting of coronary artery restenosis, cerebral artery restenosis, carotid artery restenosis, renal artery restenosis, femoral artery restenosis, peripheral artery restenosis or combinations thereof, and
(c) the proliferative disease is selected from the group consisting of hyperlasias, endometriosis, hypertrophic scars and keloids, proliferative diabetic retinopathy, glomerulonephritis, proliferative, pulmonary hypertension, rheumatoid arthritis, arteriovenous malformations, atherosclerotic plaques, delayed wound healing, hemophilic joints, nonunion fractures, Osler-Weber syndrome, psoriasis, pyogenic granuloma, scleroderma, tracoma, menorrhagia, vascular adhesions, and papillomas.
(d) neurodegenerative disease is selected from the group consisting of Alzheimer's, Pakinson's, ALS, and spinal and bulbar muscular atrophy.

20. The method of claim 19, wherein the miRNA is selected from the group consisting of:

| | |
|---|---|
| miR145 | |
| (GUCCAGUUUUCCCAGGAAUCCCUU), | (SEQ ID NO: 1) |
| miR454-3p | |
| (UAGUGCAAUAUUGCUUAUAGGGUUU), | (SEQ ID NO: 2) |
| miR519a | |
| (AAAGUGCAUCCUUUUAGAGUGUUAC), | (SEQ ID NO: 3) |
| miR520c | |
| (AAAGUGCUUCCUUUUAGAGGGUU), | (SEQ ID NO: 4) |
| miR520d | |
| (AAAGUGCUUCUCUUUGGUGGGUU), | (SEQ ID NO: 5) |
| miR-425-3p | |
| (AUCGGGAAUGUCGUGUCCGCC), | (SEQ ID NO: 6) |
| miR-495 | |
| (AAACAAACAUGGUGCACUUCUUU), | (SEQ ID NO: 7) |
| miR-572 | |
| (GUCCGCUCGGCGGUGGCCCA), | (SEQ ID NO: 8) |
| miR-661 | |
| (UGCCUGGGUCUCUGGCCUGCGCGU), | (SEQ ID NO: 9) |

complements thereof and combinations thereof.

21. The method of claim 19, wherein the miRNA is selected from the group consisting of: miR454-3p (UAGUGCAAUAUUGCUUAUAGGGUUU) (SEQ ID NO:2), miR520c (AAAGUGCUUCCUUUUAGAGGGUU) (SEQ ID NO:4), complements thereof and combinations thereof.

22. The method of claim 21, wherein the therapeutic agent is 17-AAG, oxaliplatin or a combination thereof.

23. The method of claim 19, wherein the miRNA is selected from the group consisting of:
miR-425-3p (AUCGGGAAUGUCGUGUCCGCC) (SEQ ID NO:6),
miR-495 (AAACAAACAUGGUGCACUUCUUU) (SEQ ID NO:7),
miR-572 (GUCCGCUCGGCGGUGGCCCA) (SEQ ID NO:8),
miR-661 (UGCCUGGGUCUCUGGCCUGCGCGU) (SEQ ID NO:9), complements thereof and combinations thereof.

24. The method of claim 23, wherein the therapeutic agent is paclitaxel.

25. The method of claim 22, wherein the miRNA is one or more of SEQ ID NOs:10-35.

26. The method of claim 19, wherein the organism is a human patient undergoing one or more cancer therapies selected from the group consisting of surgery, chemotherapy, radiotherapy, thermotherapy, immunotherapy, hormone therapy and laser therapy.

27. The method of claim 19, wherein the organism is a human patient undergoing one or more antiproliferative therapies consisting of surgery, chemotherapy, radiotherapy, thermotherapy, immunotherapy, hormone therapy, laser therapy, or stenting.

28. The method of claim 24, wherein the organism is a human.

29. A therapeutic composition for enhancing the activity of a therapeutic agent in an organism afflicted with cancer, neurodegenerative diseases, restenosis or proliferative cellular diseases comprising an effective amount of an miRNA or a vector that expresses an effective amount of an miRNA before, during or after administering the therapeutic agent.

30. The composition of claim 29 wherein the miRNA is selected from the group consisting of a pri-miRNA, pre-miRNA, mature miRNA, ds miRNA and fragments or variants thereof.

31. The composition of claim 29 wherein the miRNA is encoded by an isolated nucleic acid vector comprising one or more in vivo expression control elements.

32. The composition of claim 31, wherein the isolated nucleic acid has been transfected into the cells of the organism.

33. The composition of claim 29, wherein the miRNA is a naked synthetic RNA.

34. The composition of claim 29, wherein the miRNA is a synthetic chemically modified RNA.

35. The composition of claim 34, wherein the synthetic miRNA is modified with a chemical moiety selected from the group consisting of phosphorothioate, boranophosphate, 2′-O-methyl, 2′-fluoro, PEG, terminal inverted-dT base, and combinations thereof.

36. The composition of claim 29, wherein the miRNA is carried in a liposome, polymer-based nanoparticle, cholesterol conjugate, cyclodextran complex, polyethylenimine polymer or a protein complex.

37. The composition of claim 29, wherein the miRNA is for administration directly to the diseased tissue, intravenously, subcutaneously, intramuscularly, nasally, intraperitonealy, vaginally, anally, orally, intraocularly or intrathecally.

38. The composition of claim 29, wherein the miRNA is from 18 nucleotides to 170 nucleotides in length.

39. The composition of claim 38, wherein the miRNA is from 18 to 25 nucleotides in length.

40. The composition of claim 29, wherein
(a) the cancer is selected from the group consisting of circinoma in situ, atypical hyperplasia, carcinoma, sarcoma, carcinosarcoma, lung cancer, pancreatic cancer, skin cancer, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer,
(b) the restenosis is selected from the group consisting of coronary artery restenosis, cerebral artery restenosis, carotid artery restenosis, renal artery restenosis, femoral artery restenosis, peripheral artery restenosis or combinations thereof, and
(c) the proliferative disease is selected from the group consisting of hyperlasias, endometriosis, hypertrophic scars and keloids, proliferative diabetic retinopathy, glomerulonephritis, proliferative, pulmonary hypertension, rheumatoid arthritis, arteriovenous malformations, atherosclerotic plaques, delayed wound healing, hemophilic joints, nonunion fractures, Osler-Weber syndrome, psoriasis, pyogenic granuloma, scleroderma, tracoma, menorrhagia, vascular adhesions, and papillomas.
(d) neurodegenerative disease is selected from the group consisting of Alzheimer, Parkinson, ALS, and spinal and bulbar muscular atrophy.

41. The composition of claim 40, wherein the therapeutic agent is selected is a radionuclide, cancer chemotherapeutic agent, targeted anticancer agent, DNA interacalating/damaging agent, cell cycle check point inhibitor, anti-metabolites, HSP inhibitor, antibiotic, kinase inhibitor, radionuclide, biologically active polypeptide, antibody, lectin, toxin, hormone, matrix metalloproteinase inhibitors, angiostatic steroid or combinations thereof.

42. The composition of claim 40, wherein the therapeutic agent is selected from the group consisting of ¹³¹I, ⁹⁰Y, ¹¹¹In, ²¹¹At, ³²P, genistein, adriamycin, ansamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin, sirolimus, mitomycin, mitotane, mitoxantrone, nitrosurea, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, transplatinum, bleomycin, hormones, tamoxifen, diethylstilbestrol, axitinib, avastin, marimastat, bevacizumab, carboxyamidotriazole, TNP-470, CM101, IFN-α, IL-12, platelet factor-4, suramin, SU5416, thrombospondin, VEGFR antagonists, cartilage-derived angiogenesis inhibitory factor, angiostatin, endostati, 2-methoxyestradiol, tecogalan, thrombospondin, prolactin, αvβ3 inhibitors, tecogalan, BAY 12-9566, AG3340, CGS27023A, COL-3, vitaxin, ZD0101, TNP-40, thalidomide, squalamine, IM862, PTK787, fumagillin, analogues of fumagillin, BB-94, BB-2516 linomid, 17-AAG, oxaliplatin, paclitaxel and combinations thereof.

43. The composition of claim 42, wherein the therapeutic agent is 17-AAG, oxaliplatin, paclitaxel or a combination thereof.

44. The composition of claim 42 wherein the miRNA comprises a sequence selected from the group consisting of:

| | |
|---|---|
| (a) miR145 | |
| (GUCCAGUUUUCCCAGGAAUCCCUU), | (SEQ ID NO: 1) |
| miR454-3p | |
| (UAGUGCAAUAUUGCUUAUAGGGUUU), | (SEQ ID NO: 2) |
| miR519a | |
| (AAAGUGCAUCCUUUUAGAGUGUUAC), | (SEQ ID NO: 3) |
| miR520c | |
| (AAAGUGCUUCCUUUUAGAGGGUU), | (SEQ ID NO: 4) |
| miR520d | |
| (AAAGUGCUUCUCUUUGGUGGGUU), | (SEQ ID NO: 5) |
| miR-425-3p | |
| (AUCGGGAAUGUCGUGUCCGCC), | (SEQ ID NO: 6) |
| miR-495 | |
| (AAACAAACAUGGUGCACUUCUUU), | (SEQ ID NO: 7) |
| miR-572 | |
| (GUCCGCUCGGCGGUGGCCCA), | (SEQ ID NO: 8) |
| miR-661 | |
| (UGCCUGGGUCUCUGGCCUGCGCGU); | (SEQ ID NO: 9) |

(b) a RNA complementary of any of the sequences in (a); and
(c) a RNA with a sequence at least about 81% identical to 21 contiguous nucleotides of (a) or (b).

45. The method of claim 42, wherein the miRNA is selected from the group consisting: miR454-3p (UAGUGCAAUAUUGCUUAUAGGGUUU) (SEQ ID NO: 2), miR520c (AAAGUGCUUCCUUUUAGAGGGUU) (SEQ ID NO: 4), complements thereof and combinations thereof.

46. The method of claim 45, wherein the therapeutic agent is 17-AAG, oxaliplatin or a combination thereof.

47. The method of claim 42, wherein the miRNA is selected from the group consisting:

| | |
|---|---|
| miR-425-3p | |
| (AUCGGGAAUGUCGUGUCCGCC), | (SEQ ID NO: 6) |
| miR-495 | |
| (AAACAAACAUGGUGCACUUCUUU), | (SEQ ID NO: 7) |
| miR-572 | |
| (GUCCGCUCGGCGGUGGCCCA), | (SEQ ID NO: 8) |
| miR-661 | |
| (UGCCUGGGUCUCUGGCCUGCGCGU), | (SEQ ID NO: 9) |

complements thereof and combinations thereof.

48. The method of claim 47, wherein the therapeutic agent is paclitaxel.

49. A probe comprising a nucleic acid or peptidenucleic acid complementary to any of the RNA sequences of claim 44.

50. A biochip comprising the nucleic acid of peptidenucleic acid probes comprising any of the miRNA sequences of claim 49.

51. A method for predicting response to therapy with a HSP90 inhibitor, a microtubule inhibitor, or a DNA replication inhibitor:
(a) providing a biological sample of diseased tissue;
(b) measuring the level of a RNA in biological sample of diseased tissue wherein the RNA measured is selected from the group consisting of

| | |
|---|---|
| (i) miR145 | |
| (GUCCAGUUUUCCCAGGAAUCCCUU), | (SEQ ID NO: 1) |
| miR454-3p | |
| (UAGUGCAAUAUUGCUUAUAGGGUUU), | (SEQ ID NO: 2) |
| miR519a | |
| (AAAGUGCAUCCUUUUAGAGUGUUAC), | (SEQ ID NO: 3) |
| miR520c | |
| (AAAGUGCUUCCUUUUAGAGGGUU), | (SEQ ID NO: 4) |
| miR520d | |
| (AAAGUGCUUCUCUUUGGUGGGUU); | (SEQ ID NO: 5) |

(ii) an RNA complementary of (i); and
(iii) an RNA with a sequence at least about 81% identical to 21 contiguous nucleotides of (i) or (ii);
(c) comparing the level of RNA from (b) in diseased tissue with the level of the same RNA in a control, wherein a level of the nucleic acid higher than a control is indicative response to the therapy and lower than that of control is indicative of non-response to the therapy.

52. The method of claim 51, wherein the HSP90 inhibitor is 17-AAG, the microtubule inhibitor is paclitaxel, and the DNA replication inhibitor is oxaliplatin.

53. The method of claim 51, wherein the RNA is measured by RT-PCR, microarray, invader, mass spectroscopy, hybridization or TMA.

54. A method for inhibiting the expression of one or more proteins comprising administering to the organism an effective amount of one or more miRNAs from the group consisting of miR145 (GUCCAGUUUUCCCAGGAAUCCCUU) (SEQ ID NO: 1),
miR454-3p (UAGUGCAAUAUUGCUUAUAGGGUUU) (SEQ ID NO:2),
miR519a (AAAGUGCAUCCUUUUAGAGUGUUAC) (SEQ ID NO:3),
miR520c (AAAGUGCUUCCUUUUAGAGGGUU) (SEQ ID NO:4), and
miR520d (AAAGUGCUUCUCUUUGGUGGGUU) (SEQ ID NO:5).

55. The method of claim 54, wherein the protein is selected from the group consisting of FAK, CDC27, MAPK activated protein kinase 2, PAR4, PKC gamma, and RAF.

56. A method for the enhancing the expression of one or more proteins in an organism comprising administering to the organism an effective amount of one or more miRNAs from the group consisting of

| | |
|---|---|
| miR145 | |
| (GUCCAGUUUUCCCAGGAAUCCCUU), | (SEQ ID NO: 1) |
| miR454-3p | |
| (UAGUGCAAUAUUGCUUAUAGGGUUU), | (SEQ ID NO: 2) |
| miR519a | |
| (AAAGUGCAUCCUUUUAGAGUGUUAC), | (SEQ ID NO: 3) |
| miR520c | |
| (AAAGUGCUUCCUUUUAGAGGGUU), | (SEQ ID NO: 4) |
| and | |
| miR520d | |
| (AAAGUGCUUCUCUUUGGUGGGUU). | (SEQ ID NO: 5) |

57. The method of claim 56, wherein the protein is selected from the group consisting of cytokeratin 4, S100 b, and vinculin.

58. An isolated nucleic acid comprising one or more in vivo expression control elements operatively linked to a reporter gene, wherein said reporter gene is upstream of all or a portion of a 3′ untranslated region of a target gene, wherein upon transfection of the isolated nucleic acid into eukaryotic cells, the in vivo expression control elements result the production of an mRNA encoding the reporter upstream of the 3′ untranslated region of the target gene's mRNA.

59. The isolated nucleic acid of claim 58, wherein the isolated nucleic acid is a vector selected from the group consisting of a plasmid, cosmid, phagemid, virus, and artificial chromosome.

60. The isolated nucleic acid of claim 59, wherein the one or more in vivo expression control elements are selected from the group consisting of a promoter, enhancer, RNA splicing signal, and combinations thereof.

61. The isolated nucleic acid of claim 59, wherein the reporter gene encodes a luciferase protein.

62. The isolated nucleic acid of claim 59, wherein the target gene is CD44, CDC27, MAPK activated kinase 2, PAR4, or PKC gamma.

63. Method of identifying gene expression modulators comprising:
(a) transfecting eukaryotic cells with an isolated nucleic acid comprising one or more in vivo expression control elements operatively linked to a reporter gene which is cloned upstream of all or a portion of a target gene 3′ untranslated region, wherein the in vivo expression control elements result the production of an mRNA encoding the reporter upstream of the 3′ untranslated region, and
(b) transfecting other eukaryotic cells with isolated nucleic acid comprising said one or more in vivo expression control elements operatively linked to said reporter gene, wherein the expression control elements result in the transcription of an mRNA encoding the reporter molecule,
(c) contacting and mock-contacting the transfected cells from (a) and (b) with a candidate expression modulator, and
(d) comparing the reporter gene activity in the transfected cells from (a) and (b) with and without contacting the transfected cells with candidate expression modulator.

64. The method of claim 63, further comprising the co-transfection of the cells in (a) and (b), with a second report construct expressing a second reporter for the normalization the data compared in (d).

65. The method of claim 64, further comprising mutating the target gene's 3′ untranslated sequence in the reporter expression construct, transfecting said mutated reporter expression construct into eukaryotic cells, and comparing the reporter gene activity resulting from expression of the mutated and unmutated reporter expression constructs with and without contacting the transfected cells with candidate expression modulator.

66. The method of claim 65, wherein the target gene is CD44, CDC27, MAPK activated kinase 2, PAR4, and PKC gamma.

67. A kit for the identification of expression modulators comprising:
(a) first isolated nucleic acid with a first set of one or more in vivo expression control elements operatively linked to a first reporter gene which is cloned upstream of all or a portion of a 3′ untranslated region of a target gene, wherein upon transfection of said first isolated nucleic acid into eukaryotic cells, the first set of in vivo expression control elements result the production of an mRNA encoding the first reporter upstream of the target gene 3′ untranslated region;
(b) a second isolated nucleic acid comprising said the set of in vivo expression control elements from (a) operatively linked to said first reporter gene, wherein upon transfection of said second isolated nucleic acid into eukaryotic cells, the in vivo expression control elements result in the transcription of an mRNA encoding said first reporter molecule; and
(c) a third isolated nucleic acid comprising a second set of one or more in vivo expression control elements operatively linked to a second reporter gene, wherein upon transfection of the isolated nucleic acid into eukaryotic cells, said second set of in vivo expression control elements result in the expression of said second reporter.

68. The kit of claim 67, wherein the target gene is CD44, CDC27, MAPK activated kinase 2, PAR4, or PKC gamma.

69. An isolated nucleic acid comprising a miRNA, wherein when the miRNA is administered to mammalian cells and the mammalian cells are then exposed to a therapeutic agent, the mammalian cells produce 485/538 nm ratio of at least about 200 in an Apo-ONE® Homogeneous Caspase-3/7 Assay.

70. The isolated nucleic acid of claim 69, wherein the therapeutic agent is 17-AAG, oxaliplatin, paclitaxel and combinations thereof.

71. An isolated nucleic acid capable of expressing a transcript comprising a miRNA, wherein when the miRNA expressed in mammalian cells and the mammalian cells are then exposed to a therapeutic agent, the mammalian cells produce 485/538 nm ratio of at least about 200 in an Apo-ONE® Homogeneous Caspase-3/7 Assay.

72. The isolated nucleic acid of claim 71, wherein the therapeutic agent is 17-AAG, oxaliplatin, paclitaxel and combinations thereof.

73. A method for enhancing the activity of raprmycin In an organism afflicted with cancer, neurodegenerative diseases, restenosis or proliferative cellular diseases comprising administering an effective amount of a composition comprising an miRNA before, during or after administering the therapeutic agent.

74. The method of claim 73, wherein the miRNA is selected from the group consisting of a pri-miRNA, pre-miRNA, mature miRNA, ds miRNA and fragments or variants thereof.

75. The method of claim 74, wherein the miRNA is encoded by an isolated nucleic acid.

76. The method of claim 75, wherein the isolated nucleic acid is integrated into a vector.

77. The method of claim 76, wherein the vector is selected from the group consisting of a plasmid, cosmid, phagemid, virus, and artificial chromosome.

78. The method of claim 77, wherein the vector further comprises one or more in vivo expression control elements.

79. The method of claim 78, wherein the one or more in vivo expression element is selected from the group consisting of a promoter, enhancer, RNA splice sites, and combinations thereof.

80. The methods of claim 79, wherein the isolated nucleic acid is transfected into the cells of the organism.

81. The method of claim 73, wherein the miRNA is a naked synthetic RNA.

82. The method of claim 73, wherein the miRNA is a chemically modified synthetic RNA.

83. The method of claim 81, wherein the synthetic RNA is modified with a chemical moiety selected from the group consisting of phosphorothioate, boranophosphate, 2′-O-methyl, 2′-fluoro, PEG, terminal inverted-dT base, and combinations thereof.

84. The method of claim 73, wherein the miRNA is administered in a liposome, polymer-based nanoparticle, cholesterol conjugate, cyclodextran complex, polyethylenimine polymer or a protein complex.

85. The method of claim 73, wherein the miRNA is administered directly to the diseased tissue in the organism, intravenously, subcutaneously, intramuscularly, nasally, intraperitonealy, vaginally, anally, orally, intraocularly or intrathecally.

86. The method of claim 73, wherein the miRNA is from 18 nucleotides to 170 nucleotides in length.

87. The method of claim 86, wherein the miRNA is from 18 to 25 nucleotides in length.

88. The method of claim 87, wherein the miRNA is selected from the group consisting of:
CCAGUAUUAACUGUGCUGCUGA (SEQ ID NO:36),
AAGUGUGCAGGGCACUGGU (SEQ ID NO:37),
AAGGAGCUUACAAUCUAGCUGGG (SEQ ID NO:38), and combinations thereof.

89. The method of claim 19, wherein the miRNA is selected from the group consisting of SEQ ID NOs:10-35.

90. The method of claim 1, wherein the miRNA is selected from the group consisting of SEQ ID NOs: 10-35.

91. The composition of claim 29, wherein the miRNA is selected from the group consisting of SEQ ID NOs:10-35.


SEQUENCE LISTING (38 sequences)

SEQ ID NO: 1 - RNA, 24 nt, Homo sapiens
guccaguuuucccaggaaucccuu

SEQ ID NO: 2 - RNA, 25 nt, Homo sapiens
uagugcaauauugcuuauaggguuu

SEQ ID NO: 3 - RNA, 25 nt, Homo sapiens
aaagugcauccuuuuagaguguuac

SEQ ID NO: 4 - RNA, 23 nt, Homo sapiens
aaagugcuuccuuuuagaggguu

SEQ ID NO: 5 - RNA, 23 nt, Homo sapiens
aaagugcuucucuuugguggguu

SEQ ID NO: 6 - RNA, 22 nt, Homo sapiens
aucgggaaugucguguccgccc

SEQ ID NO: 7 - RNA, 23 nt, Homo sapiens
aaacaaacauggugcacuucuuu

SEQ ID NO: 8 - RNA, 20 nt, Homo sapiens
guccgcucggcgguggccca

SEQ ID NO: 9 - RNA, 24 nt, Homo sapiens
ugccugggucucuggccugcgcgu

SEQ ID NO: 10 - RNA, 22 nt, Homo sapiens
aaagugcuucucuuuggugggu

SEQ ID NO: 11 - RNA, 23 nt, Homo sapiens
guccaguuuucccaggaaucccu

SEQ ID NO: 12 - RNA, 21 nt, Homo sapiens
aucgggaaugucguguccgcc

SEQ ID NO: 13 - RNA, 22 nt, Homo sapiens
aaacaaacauggugcacuucuu

SEQ ID NO: 14 - RNA, 22 nt, Homo sapiens
aaagugcauccuuuuagagugu

SEQ ID NO: 15 - RNA, 23 nt, Homo sapiens
uagugcaauauugcuuauagggu

SEQ ID NO: 16 - RNA, 20 nt, Homo sapiens
guccgcucggcgguggccca

SEQ ID NO: 17 - RNA, 22 nt, Homo sapiens
aaagugcuuccuuuuagagggu

SEQ ID NO: 18 - RNA, 24 nt, Homo sapiens
ugccugggucucuggccugcgcgu

SEQ ID NO: 19 - RNA, 23 nt, Homo sapiens
aaugacacgaucacucccguuga

SEQ ID NO: 20 - RNA, 23 nt, Homo sapiens
aaugacacgaucacucccguuga

SEQ ID NO: 21 - RNA, 19 nt, Homo sapiens
aagugugcagggcacuggu

SEQ ID NO: 22 - RNA, 22 nt, Homo sapiens
uucaccaccuucuccacccagc

SEQ ID NO: 23 - RNA, 24 nt, Homo sapiens
aauccuuggaaccuaggugugagu

SEQ ID NO: 24 - RNA, 22 nt, Homo sapiens
aaguucuguuauacacucaggc

SEQ ID NO: 25 - RNA, 22 nt, Homo sapiens
aacaauauccuggugcugagug

SEQ ID NO: 26 - RNA, 22 nt, Homo sapiens
augguuccgucaagcaccaugg

SEQ ID NO: 27 - RNA, 22 nt, Homo sapiens
agaauuguggcuggacaucugu

SEQ ID NO: 28 - RNA, 22 nt, Homo sapiens
caguuaucacagugcugaugcu

SEQ ID NO: 29 - RNA, 21 nt, Homo sapiens
agggagggacgggggcugugc

SEQ ID NO: 30 - RNA, 23 nt, Homo sapiens
aaggagcuuacaaucuagcuggg

SEQ ID NO: 31 - RNA, 23 nt, Homo sapiens
aagugccgccaucuuuugagugu

SEQ ID NO: 32 - RNA, 23 nt, Homo sapiens
agguuguccguggugaguucgca

SEQ ID NO: 33 - RNA, 23 nt, Homo sapiens
aaaagugcuuacagugcagguag

SEQ ID NO: 34 - RNA, 22 nt, Homo sapiens
aaauuauuguacaucggaugag

SEQ ID NO: 35 - RNA, 26 nt, Homo sapiens
gugagggcaugcaggccuggaugggg

SEQ ID NO: 36 - RNA, 22 nt, Homo sapiens
ccaguauuaacugugcugcuga

SEQ ID NO: 37 - RNA, 19 nt, Homo sapiens
aagugugcagggcacuggu

SEQ ID NO: 38 - RNA, 23 nt, Homo sapiens
aaggagcuuacaaucuagcuggg